SEQUENCE LISTING (26 sequences)

SEQ ID NO: 1 - protein, 8 aa, synthetic construct
GFTFDDYA

SEQ ID NO: 2 - protein, 8 aa, synthetic construct
ISWNSGSI

SEQ ID NO: 3 - protein, 16 aa, synthetic construct
AKDSRGYGDYRLGGAY

SEQ ID NO: 4 - protein, 6 aa, synthetic construct
QSVSSN

SEQ ID NO: 5 - skipped

SEQ ID NO: 6 - protein, 9 aa, synthetic construct
QQYNNWPWT

SEQ ID NO: 7 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 8 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 9 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 107 aa, synthetic construct [sequence omitted]

SEQ ID NO: 11 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 15 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 17 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 20 - protein, 345 aa, synthetic construct
EVQLVESGGGLVQPGGSLRLSCAASGFSFSDFWMSWVRQAPGKGLEWVATISQAGSEKDYVDSVKGRFTISRDNAKKSLYLQMNSLRAEDTAVYYCASGVYSFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 21 - protein, 8 aa, synthetic construct
GFSFSDFW

SEQ ID NO: 22 - protein, 8 aa, synthetic construct
ISQAGSEK

SEQ ID NO: 23 - protein, 9 aa, synthetic construct
ASGVYSFDY

SEQ ID NO: 24 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 5 aa, synthetic construct
GGGGS

SEQ ID NO: 26 - protein, 10 aa, synthetic construct
GGGGSGGGGS

(19) United States
(12) Patent Application Publication
(10) Pub. No.: US 2026/0174866 A1
(43) Pub. Date: Jun. 25, 2026

(54) METHODS OF TREATING NON-HODGKIN LYMPHOMA

(71) Applicant: TENEOTWO, INC., Newark, CA (US)

(72) Inventors: Ben BUELOW, Newark, CA (US); Ute SCHELLENBERGER, Newark, CA (US); Ashwin PATEL, Newark, CA (US); Alessandra FORCINA, Newark, CA (US); Robert CHEN, Newark, CA (US); Makhdum AHMED, Newark, CA (US); Anas YOUNES, Newark, CA (US); Robyn LESLIE, Newark, CA (US); Karthick VISHWANATHAN, Newark, CA (US)

(21) Appl. No.: 19/126,026

(22) PCT Filed: Oct. 31, 2023
(86) PCT No.: PCT/US2023/036466

§ 371 Date: Apr. 30, 2025

Related U.S. Application Data
(60) Provisional application No. 63/381,886, filed on Nov. 1, 2022.
(60) Provisional application No. 63/504,179, filed on May 24, 2023.

(51) Int. Cl. A61K 47/10; A61K 38/00

(52) U.S. Cl. CPC A61K 47/10; A61K 38/00

(57) ABSTRACT

Methods of treating non-Hodgkin lymphoma by administering a multispecific antibody to a patient in need are provided. Methods of making such antibodies, and compositions, including pharmaceutical compositions, comprising such antibodies, are also provided.

### CROSS-REFERENCE TO RELATED APPLICATIONS

This application claims benefit under 35 U.S.C. § 119(e) of the U.S. Provisional Application Nos. 63/381,886, filed Nov. 1, 2022 and 63/504,179, filed May 24, 2023, both of which are incorporated by reference herein in their entirety for all purposes.

### REFERENCE TO THE SEQUENCE LISTING

This application incorporates by reference a Sequence Listing submitted with this application as HTML file entitled CD19TCE-201-WO-PCT, created on Oct. 31, 2023, and having a size of 28.0 kilobytes.

### FIELD

The present disclosure provides methods of treating non-Hodgkin lymphoma by administering a multi-specific (e.g. bispecific) antibody to a patient in need. The present disclosure further provides methods of making such antibodies, and compositions, including pharmaceutical compositions, comprising such antibodies.

### BACKGROUND

The rate of new Non-Hodgkin lymphoma (NHL) cases was 19.6 per 100,000 men and women per year. The death rate was 5.4 per 100,000 men and women per year. These rates are age-adjusted and based on 2014-2018 cases and deaths. The estimated new cases for 2021 is 81,560 individuals; 4.3% of all new cancer cases. The estimated new deaths for 2021 is 20,720 individuals; 3.4% of all cancer deaths. At presentation, 24% of cases are stage I defined as “confined to a single region,” 14% of cases are stage II defined as “involving multiple regions,” 16% of cases are stage III defined as “spread to both sides of diaphragm,” 33% of cases are stage IV defined as “diffuse or disseminated involvement,” and 12% are un-staged. The 5-year relative survival rates for stage I: 84.3% stage II: 77.1%, stage III 71.1%, and stage IV 63.7%. SEER database; accessed Apr. 15, 2021; https://seer.cancer.gov/.

Non-Hodgkin lymphomas include diverse neoplasms of the lymphoid compartment; in aggregate, approximately 74,000 new cases are reported each year (National Cancer Institute [NCI] Surveillance, Epidemiology and End Results [SEER]2020). In the United States (US), roughly 80% of these derive from the B-cell lineage. Among the B-cell non-Hodgkin lymphomas (B-NHL) the subtypes can be loosely grouped into ‘indolent’ lymphomas, including chronic lymphocytic leukemia/small lymphocytic lymphoma (not under investigation in this study), follicular lymphoma (FL), and marginal zone lymphoma (MZL), and the ‘aggressive’ lymphomas including diffuse large B-cell lymphoma (DLBCL), high grade B-cell lymphoma (HGBL), mantle-cell lymphoma (MCL), and transformed indolent lymphomas; additional types and subtypes exist (Swerdlow 2017).

Diagnosis often includes techniques such as physical exam, blood and urine tests, imaging, and lymph or bone marrow tests. Conventional therapies include chemotherapy administered orally or by injection, radiation therapy, and bone marrow transplants.

For DLBCL, over half of patients survive for 5 years or more using a multiagent chemotherapy regimen including rituximab, cyclophosphamide, doxorubicin, vincristine, and prednisone (e.g. R-CHOP; Friedberg 2011). Second line therapies typically consist of high dose chemotherapy followed by autologous SCT. For transplant ineligible patients, a broad range of therapies usually incorporating an anti-CD20 monoclonal antibody are used. Of these only 30 to 40% respond (Crump 2017). The prognosis for patients that do not respond to salvage therapy is poor. Third line therapies include anti-CD19 chimeric antigen receptor (CAR) T cells (e.g. Yescarta® and Kymriah®) and the anti-CD79b ADC Polivy® (Yescarta US Prescribing Information [PI]2017; Kymriah US PI 2017; Polivy US PI 2019). Eligibility for second- and third-line therapies varies widely for patients. For instance, elderly or frail patients are often not candidates for transplant, while up to 23% of relapsed or refractory (RR) DLBCL patients have disease too rapidly progressing to be eligible for CAR T-cell therapy (Paillassa 2019). Furthermore, all third line therapies described above are not considered ‘available therapies’ at this time. Beyond the second line, overall survival (OS) is in the range of 8 to 10 months (Van Den Neste 2016). Given these features, DLBCL—especially RR or high-risk disease—represents one of the most significant areas of unmet medical need in lymphoma.

Aspects of the disclosure include methods of treating this patient population.

### SUMMARY

Aspects of the disclosure include methods for treating B-cell non-Hodgkin lymphoma in a patient in need, the methods comprising administering to the patient a therapeutically effective amount of TNB-486 according to a 28-day treatment cycle, wherein the therapeutically effective amount of TNB-486 is from about 30 μg to about 30000 μg, optionally wherein the patient receives six treatment cycles.

In some aspects, the treatment cycle is repeated two or more times. In some aspects, TNB-486 is administered to the patient as a monotherapy. In some aspects, TNB-486 is administered by an intravenous infusion (IV). In some aspects, the patient has received at least two prior lines of systemic therapy. In some aspects, the patient is CD19-positive. In some aspects, the patient has an Eastern Cooperative Oncology Group (ECOG) performance status of less than or equal to 2. In some aspects, the patient has adequate bone marrow function. In some aspects, the patient has an estimated glomerular filtration rate (eGFR) greater than or equal to 50 mL/min. In some aspects, the patient has a total bilirubin of less than or equal to 1.5 times an upper limit of normal, an aspartate aminotransferase (AST) of less than or equal to 3 times an upper limit of normal, and an alanine amino transferase (ALT) of less than or equal to 3 times an upper limit of normal.

Aspects of the disclosure include methods for improving an objective response rate (ORR) in a patient diagnosed with B-cell non-Hodgkin lymphoma, the methods comprising administering to the patient a therapeutically effective amount of TNB-486 according to a 28-day treatment cycle, wherein the therapeutically effective amount of TNB-486 is from about 30 μg to about 30000 μg.

Aspects of the disclosure include methods for improving an overall survival (OS) rate in a patient diagnosed with B-cell non-Hodgkin lymphoma, the methods comprising administering to the patient a therapeutically effective amount of TNB-486 according to a 28-day treatment cycle, wherein the therapeutically effective amount of TNB-486 is from about 30 μg to about 30000 μg.

Aspects of the disclosure include methods for improving a progression free survival (PFS) rate in a patient diagnosed with B-cell non-Hodgkin lymphoma, the methods comprising administering to the patient a therapeutically effective amount of TNB-486 according to a 28-day treatment cycle, wherein the therapeutically effective amount of TNB-486 is from about 30 μg to about 30000 μg.

Aspects of the disclosure include methods for improving a time to progression (TTP) in a patient diagnosed with B-cell non-Hodgkin lymphoma, the methods comprising administering to the patient a therapeutically effective amount of TNB-486 according to a 28-day treatment cycle, wherein the therapeutically effective amount of TNB-486 is from about 30 μg to about 30000 μg.

Aspects of the disclosure include methods for improving a time to response (TTR) in a patient diagnosed with B-cell non-Hodgkin lymphoma, the methods comprising administering to the patient a therapeutically effective amount of TNB-486 according to a 28-day treatment cycle, wherein the therapeutically effective amount of TNB-486 is from about 30 μg to about 30000 μg.

Aspects of the disclosure include methods for improving a duration of objective response (DOR) in a patient diagnosed with B-cell non-Hodgkin lymphoma, the methods comprising administering to the patient a therapeutically effective amount of TNB-486 according to a 28-day cycle, wherein the therapeutically effective amount of TNB-486 is from about 30 g to about 30000 μg.

Aspects of the disclosure include methods for improving a clinical benefit rate (CBR) in a patient diagnosed with B-cell non-Hodgkin lymphoma, the methods comprising administering to the patient a therapeutically effective amount of TNB-486 according to a 28-day cycle, wherein the therapeutically effective amount of TNB-486 is from about 30 μg to about 30000 μg.

In some aspects, the improvement is at least about 5%, at least about 10%, at least about 15%, at least about 20%, at least about 25%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or at least about 100%.

In some aspects, the treatment cycle is modified to add more time between doses. In some aspects, the treatment cycle is modified by consistently eliminating one or more treatment cycles from a dosing regimen.

In some aspects, the treatment cycle is modified to include a priming dose. In some aspects, the priming dose is from about 150 μg to about 1500 μg. In some aspects, the priming dose is from about 270 μg to about 1000 μg. In some aspects, the priming dose is administered at a first timepoint in the first treatment cycle, and a full dose is administered at all subsequent timepoints. In some aspects, the priming dose is administered on day 1 of the first treatment cycle, the full dose is administered on day 15 of the first treatment cycle, and the full dose is administered on days 1 and 15 of all subsequent treatment cycles.

In some aspects, the treatment cycle is modified to include at least two priming doses. In some aspects, the first priming dose is from about 150 μg to about 540 μg. In some aspects, the second priming dose is from about 800 μg to about 1200 μg. In some aspects, the first priming dose is about 270 μg and the second priming dose is about 1000 μg. In some aspects, the first priming dose is administered on day 1 of the first treatment cycle, the second priming dose is administered on day 8 of the first treatment cycle, the full dose is administered on day 15 of the first treatment cycle, and the full dose is administered on days 1 and 15 of all subsequent treatment cycles.

In some aspects, the full dose is equal to or less than 100% more (2× more) than a full dose corresponding to a next lowest dose cohort. In some aspects, the full dose is equal to or less than 50% more than a full dose corresponding to a next lowest dose cohort. In some aspects, the full dose is equal to or less than 33% more than a full dose corresponding to a next lowest dose cohort.

In some aspects the methods further comprise a step of premedicating the patient prior to administration of TNB-486 with an agent that reduces a risk or severity of a hypersensitivity reaction. In some aspects, the agent that reduces the risk or severity of a hypersensitivity reaction is selected from the group consisting of: dexamethasone, diphenhydramine, acetaminophen, ranitidine, tocilizumab, any equivalents thereof, or any combination thereof. In some aspects, the agent that reduces the risk or severity of a hypersensitivity reaction is administered 15-60 minutes prior to administration of TNB-486. In some aspects, the therapeutically effective amount of TNB-486 is about 30 μg, 90 μg, 270 μg, 800 μg, 2400 μg, 7200 μg, 15000 μg, or 30000 μg.

In some aspects of the methods, the TNB-486 is administered in combination with another chemotherapy. In some aspects, the other chemotherapy is a combination of rituximab, cyclophosphamide, doxorubicin hydrochloride (Hydroxydaunomycin), vincristine sulfate (Oncovin), and prednisone (R-CHOP). In some aspects, the R-CHOP is administered on day 1 of the first treatment cycle and then on day 1 of each subsequent treatment cycle. In some aspects, each treatment cycle is 21 days long, R-CHOP is administered on day 1 of a first treatment cycle, a first priming dose is administered on day 8 of the first treatment cycle, a second priming dose is administered on day 15 of the first treatment cycle, and R-CHOP and a therapeutically effective amount of TNB-486 are administered on day 1 of a second treatment cycle. In some aspects, R-CHOP and a therapeutically effective amount of TNB-486 are administered on day 1 of each subsequent treatment cycle.

In some aspects, the patient has reduced cytokine release compared to a different TNB-486 dosing schedule. In some aspects, the patient does not experience cytokine release syndrome or only experiences Grade 1 cytokine release syndrome. In some aspects, the cytokine release syndrome comprises the release of IL-6 and/or TNF-α. In some aspects, the objective response rate (ORR) is 80% or above. In some aspects, the complete response (CR) rate is 90% or above. In some aspects, the 6-month PFS rate is 90% or above. The method of any previous claim, wherein TNB-486 is a bispecific molecule that binds to CD3 and human CD19, comprising: (i) a first polypeptide subunit comprising the amino acid sequence of SEQ ID NO: 18; (ii) a second polypeptide subunit comprising the amino acid sequence of SEQ ID NO: 11, wherein the first and second polypeptide subunits together form a first binding moiety that binds to human CD3; and (iii) a third polypeptide subunit that binds to human CD19, comprising the amino acid sequence of SEQ ID NO: 20.

These and further aspects will be further explained in the rest of the disclosure, including the Examples.

### BRIEF DESCRIPTION OF THE DRAWINGS

FIG. 1A is a schematic diagram showing an example method of treatment comprising a dose escalation phase (Arm A) and two dose expansion phases (Arm B and Arm C). FIG. 1B is a schematic diagram showing an example method of treatment comprising administration of TNB-486 in combination with R-CHOP (Arm D).

FIG. 2 is a schematic diagram showing an example method of treatment comprising a dose escalation phase (Arm A), and providing additional details regarding treatments for different patient cohorts.

FIG. 3 is a schematic diagram showing suggested treatment guidelines for subjects exhibiting signs and/or symptoms of cytokine release syndrome (CRS).

FIG. 4 is a schematic diagram of TNB-486, which is a three chain antibody-like molecule (TCA) comprising one arm comprising a heavy chain/light pair that binds to CD3, and a second arm comprising a heavy chain-only variable region that binds to CD19, in a monovalent configuration.

FIG. 5A is a plot of percent cytokine release syndrome incidence at different timepoints of administration as described in the Example. C1D1—cycle 1, day 1. C1D15—cycle 1, day 15. C2D1—cycle 2, day 1. The total percentage occurrence of CRS is indicated above each bar.

FIG. 5B is a plot of percent of Immune Effector Cell-Associated Neurotoxicity Syndrome (ICANS) occurrence at different timepoints of administration as described in the Example. C1D1—cycle 1, day 1. C1D15—cycle 1, day 15. C2D1—cycle 2, day 1. The total percentage occurrence of ICANS is indicated above each bar.

FIG. 6 is a schematic of patient outcomes for the subjects evaluated at the first clinical cutoff as described in the Example. Lymphoma types are as shown in the key. CR—complete response. DC-PD—discontinued, patient decision. DC-other—discontinued, other reason. PR—partial response.

FIG. 7 is a plot of the response rate for the first clinical cutoff as described in the Example. The first plot is for all types of B-Cell Hodgkin Lymphoma (B-NHL) subjects (n=25). The second plot is for the 4 subjects with diffuse large B-cell lymphoma (DLBCL). The third plot is for the 8 subjects with follicular lymphoma (FL). The overall response rate (ORR) is shown at the top of the first and second plots, with the complete response (CR) rate shown for the third plot.

FIG. 8 is a schematic of patient outcomes for the subjects evaluated at the second clinical cutoff as described in the Example. Lymphoma types are as shown in the key. CR—complete response. DC-PD—discontinued, patient decision. DC-other—discontinued, other reason. PR—partial response.

FIG. 9 shows the median cytokine level (pg/ml) for both IL-6 and TNF-α for various priming dose regimens.

FIG. 10 shows that complete response (CR) is typically achieved at the first assessment (week 8), and that the 6-month PFS rate is 91%.

### DETAILED DESCRIPTION OF THE PREFERRED ASPECTS

The practice of the present methods will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the methods disclosed herein. However, it will be apparent to one of skill in the art that the present methods may be practiced without one or more of these specific details. In other instances, well-known features and procedures well known to those skilled in the art have not been described in order to avoid obscuring the methods disclosed herein.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject methods.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VL) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or antigen-binding molecule (including heavy chain-only antibodies and multi-specific (e.g., bispecific) three-chain antibody-like molecules (TCAs), described herein) is one capable of exerting one or more of its natural activities in structural, regulatory, biochemical or biophysical events. For example, a functional antibody or other binding molecule, e.g., a TCA, may have the ability to specifically bind an antigen and the binding may in turn elicit or alter a cellular or molecular event such as signal transduction or enzymatic activity. A functional antibody or other binding molecule, e.g., a TCA, may also block ligand activation of a receptor or act as an agonist or antagonist. The capability of an antibody or other binding molecule, e.g., a TCA, to exert one or more of its natural activities depends on several factors, including proper folding and assembly of the polypeptide chains.

The term “adverse event” or “AE” is defined as any untoward medical occurrence in a clinical investigation subject administered a pharmaceutical product and which does not necessarily have a causal relationship with the treatment. An AE can therefore be any unfavorable and unintended sign (including an abnormal laboratory finding), symptom, or disease temporally associated with the use of a medicinal (investigational) product, whether or not the event is considered causally related to the use of the product.

The term “antibody” herein is used in the broadest sense and specifically covers monoclonal antibodies, polyclonal antibodies, monomers, dimers, multimers, multi-specific antibodies (e.g., bispecific antibodies), heavy chain-only antibodies, three chain antibodies, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term “antibody” may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present methods can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). In some aspects, “CDR” means a complementary determining region of an antibody as defined in Lefranc, M P et al., IMGT, the international ImMunoGeneTics database, Nucleic Acids Res., 27:209-212 (1999). “Framework Region” or “FR” residues are those variable domain residues other than the hypervariable region/CDR residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The terms “heavy chain-only antibody,” and “heavy chain antibody” are used interchangeably herein and refer, in the broadest sense, to antibodies, or more or more portions of an antibody, e.g., one or more arms of an antibody, lacking the light chain of a conventional antibody. The terms specifically include, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof, and soluble single domain antibodies (sUniDabs™). In one aspect, a heavy chain-only antibody is composed of a variable region antigen-binding domain composed of framework 1, CDR1, framework 2, CDR2, framework 3, CDR3, and framework 4. In another aspect, a heavy chain-only antibody is composed of an antigen-binding domain, at least part of a hinge region and CH2 and CH3 domains. In another aspect, a heavy chain-only antibody is composed of an antigen-binding domain, at least part of a hinge region and a CH2 domain. In a further aspect, a heavy chain-only antibody is composed of an antigen-binding domain, at least part of a hinge region and a CH3 domain. Heavy chain-only antibodies in which the CH2 and/or CH3 domain is truncated are also included herein. In a further aspect, a heavy chain is composed of an antigen binding domain, and at least one CH (CH1, CH2, CH3, or CH4) domain but no hinge region. The heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular aspect, a heavy chain antibody is of the IgG1, IgG2, IgG3, or IgG4 subtype, in particular the IgG1 subtype. In one aspect, the heavy chain-only antibodies herein are used as a binding (targeting) domain of a chimeric antigen receptor (CAR). The definition specifically includes human heavy chain-only antibodies produced by human immunoglobulin transgenic rats (UniRat™) called UniAbs™. The variable regions (VH) of UniAbs™ are called UniDabs™, and are versatile building blocks that can be linked to Fc regions or serum albumin for the development of novel therapeutics with multi-specificity, increased potency and extended half-life. Since the homodimeric UniAbs™ lack a light chain and thus a VL domain, the antigen is recognized by one single domain, i.e., the variable domain of the heavy chain of a heavy-chain antibody (VH or VHH).

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ (kappa) and λ (lambda), based on the amino acid sequences of their constant domains. Antibodies in accordance with aspects of the methods can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, such as 1, 2, 3, 4, 5, 6, 7, 8, 9 or 10 amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The human IgG4 Fc amino acid sequence (UniProtKB No. P01861) is incorporated herein. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fe, or a methionine residue is added thereto. Thus, in some aspects, one or more Fc portions of an antibody can comprise one or more mutations in the hinge region to eliminate disulfide bonding. In yet another aspect, the hinge region of an Fc can be removed entirely. In still another aspect, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

In some aspects, an antibody comprises a variant human IgG4 CH3 domain sequence comprising a T366W mutation, which can optionally be referred to herein as an IgG4 CH3 knob sequence. In some aspects, an antibody comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some aspects, an antibody comprises a heavy chain polypeptide subunit comprising a variant human IgG4 Fc region comprising an S228P mutation, an F234A mutation, an L235A mutation, and a T366W mutation (knob). In some aspects, and antibody comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this disclosure can comprise an antibody with or without K447.

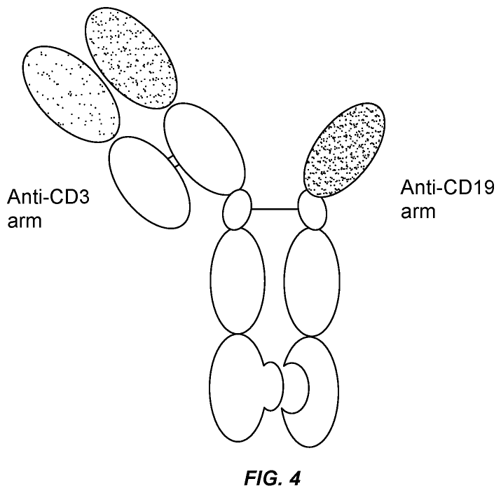

Aspects of the disclosure include antibodies comprising a heavy chain-only variable region in a monovalent or bivalent configuration. As used herein, the term “monovalent configuration” as used in reference to a heavy chain-only variable region domain means that only one heavy chain-only variable region domain is present, having a single binding site. In contrast, the term “bivalent configuration” as used in reference to a heavy chain-only variable region domain means that two heavy chain-only variable region domains are present (each having a single binding site), and are connected by a linker sequence (see FIG. 4). Non-limiting examples of linker sequences are discussed further herein, and include, without limitation, GS linker sequences of various lengths. When a heavy chain-only variable region is in a bivalent configuration, each of the two heavy chain-only variable region domains can have binding affinity to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains have binding affinity to the same target antigen.

Aspects of the disclosure include antibodies having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some aspects, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 25) (n=1) and GGGGSGGGGS (SEQ ID NO: 26) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

The term “three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

A TCA binding compound makes use of a “heavy chain only antibody” or “heavy chain antibody” or “heavy chain polypeptide” which, as used herein, mean a single chain antibody comprising heavy chain constant regions CH2 and/or CH3 and/or CH4 but no CH1 domain. In one aspect, the heavy chain antibody is composed of an antigen-binding domain, at least part of a hinge region and CH2 and CH3 domains. In another aspect, the heavy chain antibody is composed of an antigen-binding domain, at least part of a hinge region and a CH2 domain. In a further aspect, the heavy chain antibody is composed of an antigen-binding domain, at least part of a hinge region and a CH3 domain. Heavy chain antibodies in which the CH2 and/or CH3 domain is truncated are also included herein. In a further aspect, the heavy chain is composed of an antigen binding domain, and at least one CH (CH1, CH2, CH3, or CH4) domain but no hinge region. The heavy chain only antibody can be in the form of a dimer, in which two heavy chains are disulfide bonded other otherwise covalently or non-covalently attached to each other, and can optionally include an asymmetric interface between one or more of the CH domains to facilitate proper pairing between polypeptide chains. The heavy-chain antibody may belong to the IgG subclass, but antibodies belonging to other subclasses, such as IgM, IgA, IgD and IgE subclass, are also included herein. In a particular aspect, the heavy chain antibody is of the IgG1, IgG2, IgG3, or IgG4 subtype, in particular the IgG1 subtype or the IgG4 subtype. Non-limiting examples of a TCA binding compound are described in, for example, WO2017/223111 and WO2018/052503, the disclosures of which are incorporated herein by reference in their entirety.

Heavy-chain antibodies constitute about one fourth of the IgG antibodies produced by the camelids, e.g., camels and llamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The terms “CD19” and “cluster of differentiation 19” as used herein refer to a molecule expressed during all phases of B cell development until terminal differentiation into plasma cells. The term “CD19” includes a CD19 protein of any human and non-human animal species, and specifically includes human CD19 as well as CD19 of non-human mammals.

The term “human CD19” as used herein includes any variants, isoforms and species homologs of human CD19 (UniProt P15391), regardless of its source or mode of preparation. Thus, “human CD19” includes human CD19 naturally expressed by cells and CD19 expressed on cells transfected with the human CD19 gene.

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProtP04234), two CD3ε chains (SwissProt P07766), and one CD3ζ chain homodimer (SwissProt 20963), and which is associated with the T-cell receptor α and β chain. The term “CD3” includes any CD3 variant, isoform and species homolog which is naturally expressed by cells (including T-cells) or can be expressed on cells transfected with genes or cDNA encoding those polypeptides, unless noted.

A “CD19 x CD3 antibody” is a multispecific heavy chain-only antibody, such as a bispecific heavy chain-only antibody, which comprises two different antigen-binding regions, one of which binds specifically to CD19 and one of which binds specifically to CD3.

The terms “anti-CD19 heavy chain-only antibody,” “CD19 heavy chain-only antibody,” “anti-CD19 heavy chain antibody” and “CD19 heavy chain antibody” are used herein interchangeably to refer to a heavy chain-only antibody as hereinabove defined, immunospecifically binding to CD19, including human CD19, as hereinabove defined. The definition includes, without limitation, human heavy chain antibodies produced by transgenic animals, such as transgenic rats or transgenic mice expressing human immunoglobulin, including UniRats™ producing human anti-CD19 UniAb™ antibodies, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred aspects, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the present disclosure include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The terms “multi-specific antibody,” “multi-specific heavy chain-only antibody,” “multi-specific heavy chain antibody,” “multi-specific UniAb™”, and “multi-specific binding compound” are used herein in the broadest sense and cover all antibodies with more than one binding specificity. The multi-specific heavy chain anti-CD19 antibodies of the present disclosure specifically include antibodies immunospecifically binding to one single epitope on a CD19 protein, such as a human CD19, and to an epitope on a different protein, such as, for example, a CD3 protein (i.e., bivalent and monoparatopic). The multi-specific heavy chain anti-CD19 antibodies of the present disclosure specifically include antibodies immunospecifically binding to two or more non-overlapping epitopes on a CD19 protein, such as a human CD19 (i.e., bivalent and biparatopic). The multi-specific heavy chain anti-CD19 antibodies of the present disclosure also specifically include antibodies immunospecifically binding to an epitope on a CD19 protein, such as human CD19 and to an epitope on a different protein, such as, for example, a CD3 protein, such as human CD3 (i.e., bivalent and biparatopic). The multi-specific heavy chain anti-CD19 antibodies of the present disclosure also specifically include antibodies immunospecifically binding to two or more non-overlapping or partially overlapping epitopes on a CD19 protein, such as a human CD19 protein, and to an epitope on a different protein, such as, for example, a CD3 protein, such as human CD3 protein (i.e., trivalent and biparatopic).

Antibodies of the disclosure include monospecific antibodies, having one binding specificity. Monospecific antibodies specifically include antibodies comprising a single binding specificity, as well as antibodies comprising more than one binding unit having the same binding specificity. The terms “monospecific antibody,” “monospecific heavy chain-only antibody,” “monospecific heavy chain antibody,” and “monospecific UniAb™” are used herein in the broadest sense and cover all antibodies with one binding specificity. The monospecific heavy chain anti-CD19 antibodies of the present disclosure specifically include antibodies immunospecifically binding to one epitope on a CD19 protein, such as a human CD19 (monovalent and monospecific). The monospecific heavy chain anti-CD19 antibodies of the present disclosure also specifically include antibodies having more than one binding unit (e.g., multivalent antibodies) immunospecifically binding to an epitope on a CD19 protein, such as human CD19. For example, a monospecific antibody in accordance with aspects of the disclosure can include a heavy chain variable region comprising two antigen-binding domains, wherein each antigen-binding domain binds to the same epitope on a CD19 protein (i.e., bivalent and monospecific).

An “epitope” is the site on the surface of an antigen molecule to which a single antibody molecule binds. Generally, an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

The term “valent” as used herein refers to a specified number of binding sites in an antibody molecule.

A “monovalent” antibody has one binding site. Thus, a monovalent antibody is also monospecific.

A “multi-valent” antibody has two or more binding sites. Thus, the terms “bivalent”, “trivalent”, and “tetravalent” refer to the presence of two binding sites, three binding sites, and four binding sites, respectively. Thus, a bispecific antibody according to the present disclosure is at least bivalent and may be trivalent, tetravalent, or otherwise multi-valent. A bivalent antibody in accordance with aspects of the disclosure may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

The term “three-chain antibody like molecule” or “TCA” is used herein to refer to antibody-like molecules comprising, consisting essentially of, or consisting of three polypeptide subunits, two of which comprise, consist essentially of, or consist of one heavy chain and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain-only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. A TCA protein makes use of a heavy chain-only antibody as hereinabove defined.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes heavy chain-only antibodies having human heavy chain variable region sequences, produced by transgenic animals, such as transgenic rats or mice, in particular UniAbs™ produced by UniRats™, as defined above.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the present disclosure that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some aspects, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some aspects, an effector cell may phagocytose a target antigen or target cell.

“Human effector cells” are leukocytes which express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“B-Cell Hodgkin Lymphoma (B-NHL) is defined to include diffuse large B-cell lymphoma (DLBCL; including subtypes such as primary mediastinal B-cell lymphoma), high-grade B-cell lymphoma (HGBL; including subtypes such as HGBL with MYC and BCL2 and/or BCL6 rearrangements), transformed indolent NHL including Richter's transformation, mantle cell lymphoma (MCL), follicular lymphoma (FL, Grade 1-3), or marginal zone lymphoma (MZL).

“CD19-positive” is or refers to an expression of CD19 on ≥50% of tumor cells as assessed by a pathologist using immunohistochemistry (IHC) or flow cytometry.

The term “characterized by expression of CD19” broadly refers to any disease or disorder in which CD19 expression is associated with or involved with one or more pathological processes that are characteristic of the disease or disorder. Such disorders include, but are not limited to, B-cell neoplasms.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “subject,” “individual,” and “patient” are used interchangeably herein to refer to a mammal being assessed for treatment and/or being treated. In an aspect, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

Abbreviations used herein include the following: ADA (Antidrug antibody); ADT (Androgen deprivation therapy); AE (Adverse event); ALT (Alanine aminotransferase); ANC (Absolute neutrophil count); AR (Androgen receptor); AST (Aspartate aminotransferase), AUC (Area under the concentration-time curve); AUCt (Area under the serum concentration-time curve from time zero to time of last measurable concentration); CAR (Chimeric antigen receptor); CARTOX (CAR-T-cell therapy associated toxicity); CBR (Clinical benefit rate); CI (Confidence interval); CL (Clearance); Cmax (Maximum observed serum concentration); CNS (Central nervous system); CR (Complete response); CRS (Cytokine release syndrome); Css, trough (Trough concentration at steady state); CT (Computed tomography); CTCAE (Common Terminology Criteria for Adverse Events); DLT (Dose limiting toxicity); DOR (Duration of Response); ECG (Electrocardiogram); ECOG (Eastern Cooperative Oncology Group); eCRF (Electronic Case Report Form); EDC (Electronic Data Capture); eGFR (Estimated glomerular filtration rate); EOI (End of infusion); EOT (End of treatment); FIH (First-in-human); FISH (Fluorescence in situ hybridization); FL (Follicular lymphoma); GCP (Good Clinical Practice); G-CSF (Granulocyte colony-stimulating factor); GLP (Good laboratory practice); HBsAg (Hepatitis B surface antigen); HBV (Hepatitis B virus); HCV (Hepatitis C virus); HCV Ab (Hepatitis C virus antibody); HIV (Human immunodeficiency virus); IB (Investigator's Brochure); ICE (Immune effector cell encephalopathy); ICF (Informed consent form); ICH (International Conference on Harmonization); IEC (Independent Ethics Committee); IMT (Immune mediated toxicity); IRB (Institutional Review Board); IV (Intravenous); mAb (Monoclonal antibody); MABEL (Minimal anticipated biological effect level); MED (Minimum efficacious dose); MedDRA (Medical Dictionary for Regulatory Activities); MR (Minor response); MRI (Magnetic Resonance Imaging); MTD (Maximum tolerated dose); NA (Not applicable); NCA (Noncompartmental analysis); NCCN (National Comprehensive Cancer Network); NCI (National Cancer Institute); B-NHL (B-Cell Non-Hodgkin Lymphoma) NLCB (No longer clinically benefitting); NT (Neurotoxicity); ORR (Objective response rate); OS (Overall survival); PARPi (Poly-ADP-ribose-polymerase inhibitors); PBMC (Peripheral blood mononuclear cells); PC (Positive control); PD (pharmacodynamic(s) or Progressive Disease); PET (Positron emission tomography); PT (Prescribing information); PK (Pharmacokinetic); PFS (Progression-free Survival); PO (per os, orally); PR (Partial response); PT (Prothrombin time); Q3W (Once every 3 weeks); QTc (QT interval corrected for heart rate); RLT (Radioligand therapy); rPFS (Radiographic progression-free survival); RP2D (Recommended phase 2 dose); RR (Relapsed or refractory); SAE (Serious adverse event); SCT (Stem cell transplant); SD (Stable disease); SMG (Safety monitoring group); SUSAR (Suspected unexpected serious adverse reaction); T_{1/2} or t_{1/2} (Terminal phase elimination half-life); T-BsAbs (T-cell engaging bispecific antibodies); TEAE (Treatment emergent adverse event); TLS (Tumor lysis syndrome); Tmax (Time to maximum observed serum concentration); TTP (Time to progression); TTR (Time to response); ULN (Upper limit of normal); US (United States); V₁ (Central compartment volume); Vss (Volume of distribution at steady state); WHO (World Health Organization).

### II. TNB-486 (Anti-CD3/Anti-CD19)

The present disclosure relates to methods of treating non-Hodgkin lymphoma by administering a bispecific three chain antibody-like molecule (TCA) to a patient in need. In some aspects, a TCA is referred to as TNB-486, and comprises an anti-CD3 binding domain that is paired with an anti-CD19 VH binding domain as shown in FIG. 4, wherein the anti-CD3 VH domain and the anti-CD3 VL domain together have binding affinity for CD3, and the TCA further comprises a heavy chain variable domain of a heavy chain-only antibody having binding affinity to CD19, in a monovalent configuration, and the TCA further comprises a variant human IgG4 Fc domain comprising a first heavy chain constant region sequence comprising an S228P mutation, an F234A mutation, an L235A mutation, and a T366W mutation (knob), and a second heavy chain constant region sequence comprising an S228P mutation, an F234A mutation, an L235A mutation, a T366S mutation, an L368A mutation, and a Y407V mutation (hole).

In some aspects, a multi-specific antibody comprises a CD3-binding VH domain that is paired with a light chain variable domain. In certain aspects, the light chain is a fixed light chain. In some aspects, the CD3-binding VH domain comprises a CDR1 sequence of SEQ ID NO: 1, a CDR2 sequence of SEQ ID NO: 2, and a CDR3 sequence of SEQ ID NO: 3, in a human VH framework. In some aspects, the fixed light chain comprises a CDR1 sequence of SEQ ID NO: 4, a CDR2 sequence of SEQ ID NO: 5, and a CDR3 sequence of SEQ ID NO: 6, in a human VL framework. Together, the CD3-binding VH domain and the light chain variable domain have binding affinity for CD3. In some aspects, a CD3-binding VH domain comprises a heavy chain variable region sequence of SEQ ID NO: 7. In some aspects, a CD3-binding VH domain comprises a sequence having at least about 80%, at least about 85%, at least about 90%, at least about 95%, or at least about 99% percent identity to the heavy chain variable region sequence of SEQ ID NO: 7. In some aspects, a fixed light chain comprises a light chain variable region sequence of SEQ ID NO: 8. In some aspects, a fixed light chain comprises a sequence having at least about 80%, at least about 85%, at least about 90%, at least about 95%, or at least about 99% percent identity to the heavy chain variable region sequence of SEQ ID NO: 8.

Multi-specific antibodies comprising the above-described CD3-binding VH domain and light chain variable domain have advantageous properties, for example, as described in PCT Publication No. WO2018/052503, the disclosure of which is incorporated by reference herein in its entirety.

TABLE 1
Anti-CD3 Heavy and Light Chain CDR1, CDR2,
CDR3 amino acid sequences.

| | SEQ_aa_CDR1 | SEQ_aa_CDR2 | SEQ_aa_CDR3 |
|---|---|---|---|
| Heavy | GFTFDDYA | ISWNSGSI | AKDSRGYGDYRL |
| Chain | (SEQ ID | (SEQ ID | GGAY |
| | NO: 1) | NO: 2) | (SEQ ID |
| | | | NO: 3) |
| Light | QSVSSN | GAS | QQYNNWPWT |
| Chain | (SEQ ID | (SEQ ID | (SEQ ID |
| | NO: 4) | NO: 5) | NO: 6) |

TABLE 2
Anti-CD3 heavy and light chain variable
region amino acid sequences.

| | |
|---|---|
| VH | EVQLVESGGGLVQPGRSLRLSCAASGFTFDDYAMHWVRQA |
| | PGKGLEWVSGISWNSGSIGYADSVKGRFTISRDNAKNSLY |
| | LQMNSLRAEDTALYYCAKDSRGYGDYRLGGAYWGQGTLVT |
| | VSS |
| | (SEQ ID NO: 7) |
| VL | EIVMTQSPATLSVSPGERATLSCRASQSVSSNLAWYQQKP |
| | GQAPRLLIYGASTRATGIPARFSGSGSGTEFTLTISSLQS |
| | EDFAVYYCQQYNNWPWTFGQGTKVEIK |
| | (SEQ ID NO: 8) |

TABLE 3
Human IgG4 Fc region sequence with
silencing mutations.

| | |
|---|---|
| Human | ASTKGPSVFPLAPCSRSTSESTAALGCLVKDYF |
| IgG4 with | PEPVTVSWNSGALTSGVHTFPAVLQSSGLYSLS |
| silencing | SVVTVPSSSLGTKTYTCNVDHKPSNTKVDKRVE |
| mutations | SKYGPPCPPCPAPEAAGGPSVFLFPPKPKDTLM |
| (Fc | ISRTPEVTCVVVDVSQEDPEVQFNWYVDGVEVH |
| region) | NAKTKPREEQFNSTYRVVSVLTVLHQDWLNGKE |
| | YKCKVSNKGLPSSIEKTISKAKGQPREPQVYTL |
| | PPSQEEMTKNQVSLTCLVKGFYPSDIAVEWESN |
| | GQPENNYKTTPPVLDSDGSFFLYSRLTVDKSRW |
| | QEGNVFSCSVMHEALHNHYTQKSLSLSLGK |
| | (SEQ ID NO: 9) |

TABLE 4
Additional sequences.

| | |
|---|---|
| Anti-CD3 | RTVAAPSVFIFPPSDEQLKSGTASVV |
| light | CLLNNFYPREAKVQWKVDNALQSGNS |
| chain | QESVTEQDSKDSTYSLSSTLTLSKAD |
| constant | YEKHKVYACEVTHQGLSSPVTKSFNR |
| region | GEC |
| sequence | (SEQ ID NO: 10) |
| (kappa | |
| light | |
| chain) | |
| Anti-CD3 | EIVMTQSPATLSVSPGERATLSCRAS |
| full | QSVSSNLAWYQQKPGQAPRLLIYGAS |
| length | TRATGIPARFSGSGSGTEFTLTISSL |
| light | QSEDFAVYYCQQYNNWPWTFGQGTKV |
| chain | EIKRTVAAPSVFIFPPSDEQLKSGTA |
| (VL + | SVVCLLNNFYPREAKVQWKVDNALQS |
| kappa | GNSQESVTEQDSKDSTYSLSSTLTLS |
| CL) | KADYEKHKVYACEVTHQGLSSPVTKS |
| | FNRGEC |
| | (SEQ ID NO: 11) |
| Anti-CD3 | EVQLVESGGGLVQPGRSLRLSCAASG |
| heavy | FTFDDYAMHWVRQAPGKGLEWVSGIS |
| chain | WNSGSIGYADSVKGRFTISRDNAKNS |
| VH F2B | LYLQMNSLRAEDTALYYCAKDSRGYG |
| (+ wt | DYRLGGAYWGQGTLVTVSSASTKGPS |
| IgG4 Fc) | VFPLAPCSRSTSESTAALGCLVKDYF |
| | PEPVTVSWNSGALTSGVHTFPAVLQS |
| | SGLYSLSSVVTVPSSSLGTKTYTCNV |
| | DHKPSNTKVDKRVESKYGPPCPSCPA |
| | PEFLGGPSVFLFPPKPKDTLMISRTP |
| | EVTCVVVDVSQEDPEVQFNWYVDGVE |
| | VHNAKTKPREEQFNSTYRVVSVLTVL |
| | HQDWLNGKEYKCKVSNKGLPSSIEKT |
| | ISKAKGQPREPQVYTLPPSQEEMTKN |
| | QVSLTCLVKGFYPSDIAVEWESNGQP |
| | ENNYKTTPPVLDSDGSFFLYSRLTVD |
| | KSRWQEGNVFSCSVMHEALHNHYTQK |
| | SLSLSLGK |
| | (SEQ ID NO: 12) |
| Anti-CD3 | EVQLVESGGGLVQPGRSLRLSCAASG |
| heavy | FTFDDYAMHWVRQAPGKGLEWVSGIS |
| chain | WNSGSIGYADSVKGRFTISRDNAKNS |
| VH F2B | LYLQMNSLRAEDTALYYCAKDSRGYG |
| (+ | DYRLGGAYWGQGTLVTVSSASTKGPS |
| silenced | VFPLAPCSRSTSESTAALGCLVKDYF |
| IgG4 Fc) | PEPVTVSWNSGALTSGVHTFPAVLQS |
| | SGLYSLSSVVTVPSSSLGTKTYTCNV |
| | DHKPSNTKVDKRVESKYGPPCPPCPA |
| | PEAAGGPSVFLFPPKPKDTLMISRTP |
| | EVTCVVVDVSQEDPEVQFNWYVDGVE |
| | VHNAKTKPREEQFNSTYRVVSVLTVL |
| | HQDWLNGKEYKCKVSNKGLPSSIEKT |
| | ISKAKGQPREPQVYTLPPSQEEMTKN |
| | QVSLTCLVKGFYPSDIAVEWESNGQP |
| | ENNYKTTPPVLDSDGSFFLYSRLTVD |
| | KSRWQEGNVFSCSVMHEALHNHYTQK |
| | SLSLSLGK |
| | (SEQ ID NO: 13) |
| Silenced | ASTKGPSVFPLAPCSRSTSESTAALG |
| IgG4 | CLVKDYFPEPVTVSWNSGALTSGVHT |
| (CH1- | FPAVLQSSGLYSLSSVVTVPSSSLGT |
| hinge- | KTYTCNVDHKPSNTKVDKRVESKYGP |
| CH2-CH3; | PCPPCPAPEAAGGPSVFLFPPKPKDT |
| hole | LMISRTPEVTCVVVDVSQEDPEVQFN |
| (S228P, | WYVDGVEVHNAKTKPREEQFNSTYRV |
| F234A, | VSVLTVLHQDWLNGKEYKCKVSNKGL |
| L235A; | PSSIEKTISKAKGQPREPQVYTLPPS |
| T366S, | QEEMTKNQVSLSCAVKGFYPSDIAVE |
| L368A, | WESNGQPENNYKTTPPVLDSDGSFFL |
| Y407V)) | VSRLTVDKSRWQEGNVFSCSVMHEAL |
| | HNHYTQKSLSLSLGK |
| | (SEQ ID NO: 14) |
| Silenced | ASTKGPSVFPLAPCSRSTSESTAALG |
| IgG4 | CLVKDYFPEPVTVSWNSGALTSGVHT |
| (CH1- | FPAVLQSSGLYSLSSVVTVPSSSLGT |
| hinge- | KTYTCNVDHKPSNTKVDKRVESKYGP |
| CH2-CH3; | PCPPCPAPEAAGGPSVFLFPPKPKDT |
| knob | LMISRTPEVTCVVVDVSQEDPEVQFN |
| (S228P, | WYVDGVEVHNAKTKPREEQFNSTYRV |
| F234A, | VSVLTVLHQDWLNGKEYKCKVSNKGL |
| L235A; | PSSIEKTISKAKGQPREPQVYTLPPS |
| T366W)) | QEEMTKNQVSLWCLVKGFYPSDIAVE |
| | WESNGQPENNYKTTPPVLDSDGSFFL |
| | YSRLTVDKSRWQEGNVFSCSVMHEAL |
| | HNHYTQKSLSLSLGK |
| | (SEQ ID NO: 15) |
| Silenced | ESKYGPPCPPCPAPEAAGGPSVFLFP |
| IgG4 | PKPKDTLMISRTPEVTCVVVDVSQED |
| (hinge- | PEVQFNWYVDGVEVHNAKTKPREEQF |
| CH2-CH3; | NSTYRVVSVLTVLHQDWLNGKEYKCK |
| hole | VSNKGLPSSIEKTISKAKGQPREPQV |
| (S228P, | YTLPPSQEEMTKNQVSLSCAVKGFYP |
| F234A, | SDIAVEWESNGQPENNYKTTPPVLDS |
| L235A; | DGSFFLVSRLTVDKSRWQEGNVFSCS |
| T366S, | VMHEALHNHYTQKSLSLSLGK |
| L368A, | (SEQ ID NO: 16) |
| Y407V)) | |
| Silenced | ESKYGPPCPPCPAPEAAGGPSVFLFP |
| IgG4 | PKPKDTLMISRTPEVTCVVVDVSQED |
| (hinge- | PEVQFNWYVDGVEVHNAKTKPREEQF |
| CH2-CH3; | NSTYRVVSVLTVLHQDWLNGKEYKCK |
| knob | VSNKGLPSSIEKTISKAKGQPREPQV |
| (S228P, | YTLPPSQEEMTKNQVSLWCLVKGFYP |
| F234A, | SDIAVEWESNGQPENNYKTTPPVLDS |
| L235A; | DGSFFLYSRLTVDKSRWQEGNVFSCS |
| T366W)) | VMHEALHNHYTQKSLSLSLGK |
| | (SEQ ID NO: 17) |
| Anti-CD3 | EVQLVESGGGLVQPGRSLRLSCAASG |
| full | FTFDDYAMHWVRQAPGKGLEWVSGIS |
| length | WNSGSIGYADSVKGRFTISRDNAKNS |
| heavy | LYLQMNSLRAEDTALYYCAKDSRGYG |
| chain | DYRLGGAYWGQGTLVTVSSASTKGPS |
| (VH | VFPLAPCSRSTSESTAALGCLVKDYF |
| F2B + | PEPVTVSWNSGALTSGVHTFPAVLQS |
| silenced | SGLYSLSSVVTVPSSSLGTKTYTCNV |
| IgG4 | DHKPSNTKVDKRVESKYGPPCPPCPA |
| Fc + | PEAAGGPSVFLFPPKPKDTLMISRTP |
| knob | EVTCVVVDVSQEDPEVQFNWYVDGVE |
| (S228P, | VHNAKTKPREEQFNSTYRVVSVLTVL |
| F234A, | HQDWLNGKEYKCKVSNKGLPSSIEKT |
| L235A; | ISKAKGQPREPQVYTLPPSQEEMTKN |
| T366W)) | QVSLWCLVKGFYPSDIAVEWESNGQP |
| | ENNYKTTPPVLDSDGSFFLYSRLTVD |
| | KSRWQEGNVFSCSVMHEALHNHYTQK |
| | SLSLSLGK |
| | (SEQ ID NO: 18) |
| Anti-CD3 | EVQLVESGGGLVQPGRSLRLSCAASG |
| full | FTFDDYAMHWVRQAPGKGLEWVSGIS |
| length | WNSGSIGYADSVKGRFTISRDNAKNS |
| heavy | LYLQMNSLRAEDTALYYCAKDSRGYG |
| chain | DYRLGGAYWGQGTLVTVSSASTKGPS |
| (VH | VFPLAPCSRSTSESTAALGCLVKDYF |
| F2B + | PEPVTVSWNSGALTSGVHTFPAVLQS |
| silenced | SGLYSLSSVVTVPSSSLGTKTYTCNV |
| IgG4 | DHKPSNTKVDKRVESKYGPPCPPCPA |
| Fc + | PEAAGGPSVFLFPPKPKDTLMISRTP |
| hole | EVTCVVVDVSQEDPEVQFNWYVDGVE |
| (S228P, | VHNAKTKPREEQFNSTYRVVSVLTVL |
| F234A, | HQDWLNGKEYKCKVSNKGLPSSIEKT |
| L235A; | ISKAKGQPREPQVYTLPPSQEEMTKN |
| T366S, | QVSLSCAVKGFYPSDIAVEWESNGQP |
| L368A, | ENNYKTTPPVLDSDGSFFLVSRLTVD |
| Y407V)) | KSRWQEGNVFSCSVMHEALHNHYTQK |
| | SLSLSLGK |
| | (SEQ ID NO: 19) |
| CD19 | EVQLVESGGGLVQPGGSLRLSCAASG |
| mono- | FSFSDFWMSWVRQAPGKGLEWVATIS |
| valent | QAGSEKDYVDSVKGRFTISRDNAKKS |
| heavy | LYLQMNSLRAEDTAVYYCASGVYSFD |
| chain VH | YRGQGTLVTVSSESKYGPPCPPCPAP |
| (TNB- | EAAGGPSVFLFPPKPKDTLMISRTPE |
| 486) + | VTCVVVDVSQEDPEVQFNWYVDGVEV |
| silenced | HNAKTKPREEQFNSTYRVVSVLTVLH |
| IgG4 Fc | QDWLNGKEYKCKVSNKGLPSSIEKTI |
| hinge | SKAKGQPREPQVYTLPPSQEEMTKNQ |
| CH2 CH3, | VSLSCAVKGFYPSDIAVEWESNGQPE |
| hole | NNYKTTPPVLDSDGSFFLVSRLTVDK |
| (S228P, | SRWQEGNVFSCSVMHEALHNHYTQKS |
| F234A, | LSLSLGK |
| L235A, | (SEQ ID NO: 20) |
| T366S, | |
| L368A, | |
| Y407V | |
| CD19 | GFSFSDFW |
| CDR1 | (SEQ ID NO: 21) |
| CD19 | ISQAGSEK |
| CDR2 | (SEQ ID NO: 22) |
| CD19 | ASGVYSFDY |
| CDR3 | (SEQ ID NO: 23) |
| Human | ASTKGPSVFPLAPCSRSTSESTAALG |
| wt IgG4 | CLVKDYFPEPVTVSWNSGALTSGVHT |
| (Uni- | FPAVLQSSGLYSLSSVVTVPSSSLGT |
| ProtKB | KTYTCNVDHKPSNTKVDKRVESKYGP |
| No. | PCPSCPAPEFLGGPSVFLFPPKPKDT |
| P01861) | LMISRTPEVTCVVVDVSQEDPEVQFN |
| | WYVDGVEVHNAKTKPREEQFNSTYRV |
| | VSVLTVLHQDWLNGKEYKCKVSNKGL |
| | PSSIEKTISKAKGQPREPQVYTLPPS |
| | QEEMTKNQVSLTCLVKGFYPSDIAVE |
| | WESNGQPENNYKTTPPVLDSDGSFFL |
| | YSRLTVDKSRWQEGNVFSCSVMHEAL |
| | HNHYTQKSLSLSLGK |
| | (SEQ ID NO: 24) |
| Linker | GGGGS |
| | (SEQ ID NO: 25) |
| Linker | GGGGSGGGGS |
| | (SEQ ID NO: 26) |

In some aspects, bispecific or multi-specific antibodies are provided, which may have any of the configurations discussed herein, including, without limitation, a bispecific three-chain antibody like molecule. In some aspects, a bispecific antibody can comprise at least one heavy chain variable region having binding specificity for CD19, and at least one heavy chain variable region having binding specificity for a different protein, e.g., CD3. In some aspects, a bispecific antibody can comprise a heavy chain/light chain pair that has binding specificity for a first antigen, and a heavy chain from a heavy chain-only antibody, comprising an Fc portion comprising CH2 and/or CH3 and/or CH4 domains, in the absence of a CH1 domain, and an antigen binding domain that binds an epitope of a second antigen or a different epitope of the first antigen, in a monovalent or bivalent configuration. In one particular aspect, a bispecific antibody comprises a heavy chain/light chain pair that has binding specificity for an antigen on an effector cell (e.g., a CD3 protein on a T-cell), and a heavy chain from a heavy chain-only antibody comprising an antigen-binding domain that has binding specificity for CD19, in a monovalent or bivalent configuration.

In some aspects, where an antibody of the present disclosure is a bispecific antibody, one arm of the antibody (one binding moiety, or one binding unit) is specific for human CD19, while the other arm may be specific for target cells, tumor-associated antigens, targeting antigens, e.g., integrins, etc., pathogen antigens, checkpoint proteins, and the like. Target cells specifically include cancer cells. In some aspects, one arm of the antibody (one binding moiety, or one binding unit) is specific for human CD19, while the other arm is specific for CD3.

In one preferred aspect, an antibody is a bispecific TCA comprising a first polypeptide comprising SEQ ID NO: 11, a second polypeptide comprising SEQ ID NO: 18, and a third polypeptide comprising SEQ ID NO: 20. In some aspects, CDR1 comprises a polypeptide comprising SEQ ID NO: 21. In some aspects, CDR2 comprises a polypeptide comprising SEQ ID NO: 22. In some aspects, CDR3 comprises a polypeptide comprising SEQ ID NO: 23. This antibody is also referred to TNB-486, and is also described in U.S. Pat. No. 11,390,681B2 and PCT publication WO2021/222578, the disclosures of each of which are incorporated by reference herein in their entirety.

### III. Preparation of Antibodies

The multispecific antibodies of the present disclosure can be prepared by methods known in the art. In a preferred aspect, the heavy chain antibodies herein are produced by transgenic animals, including transgenic mice and rats, preferably rats, in which the endogenous immunoglobulin genes are knocked out or disabled. In a preferred aspect, the heavy chain antibodies herein are produced in UniRat™. UniRat™ have their endogenous immunoglobulin genes silenced and use a human immunoglobulin heavy-chain translocus to express a diverse, naturally optimized repertoire of fully human HCAbs. While endogenous immunoglobulin loci in rats can be knocked out or silenced using a variety of technologies, in UniRat™ the zinc-finger (endo)nuclease (ZNF) technology was used to inactivate the endogenous rat heavy chain J-locus, light chain Cκ locus and light chain Cλ locus. ZNF constructs for microinjection into oocytes can produce IgH and IgL knock out (KO) lines. For details see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy chain antibodies produced in UniRat™ are called UniAbs™ and can bind epitopes that cannot be attacked with conventional antibodies. Their high specificity, affinity, and small size make them ideal for mono- and poly-specific applications.

In addition to UniAbs™, specifically included herein are heavy chain-only antibodies lacking the camelid VHH framework and mutations, and their functional VH regions. Such heavy chain-only antibodies can, for example, be produced in transgenic rats or mice which comprise fully human heavy chain-only gene loci as described, e.g., in WO2006/008548, but other transgenic mammals, such as rabbit, guinea pig, rat can also be used, rats and mice being preferred. Heavy chain-only antibodies, including their VHH or VH functional fragments, can also be produced by recombinant DNA technology, by expression of the encoding nucleic acid in a suitable eukaryotic or prokaryotic host, including, for example, mammalian cells (e.g., CHO cells), E. coli or yeast.

Domains of heavy chain-only antibodies combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of HCAbs can be manufactured in a cost-effective manner.

In a particular aspect, the heavy chain antibodies of the present disclosure, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in HCAbs and are, at least partially, for the unwanted aggregation and light chain association of HCAbs. The substituted amino acid residue preferably is charged, and more preferably is positively charged, such as lysine (Lys, K), arginine (Arg, R) or histidine (His, H), preferably arginine (R). In a preferred aspect the heavy chain-only antibodies derived from the transgenic animals contain a Trp to Arg mutation at position 101. The resultant HCAbs preferably have high antigen-binding affinity and solubility under physiological conditions in the absence of aggregation.

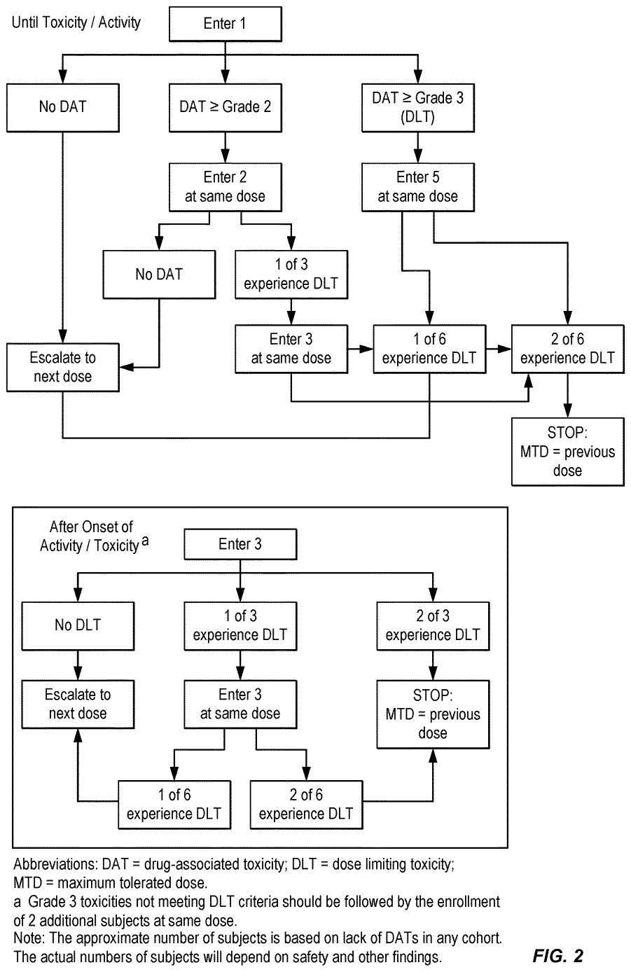

As part of the present disclosure, human IgG anti-CD19 heavy chain antibodies with unique sequences from UniRat™ animals (UniAb™) were identified that bind human CD19 in ELISA (recombinant CD19 extracellular domain) protein and cell-binding assays. The identified heavy chain variable region (VH) sequences (see FIG. 2) are positive for human CD19 protein binding and/or for binding to CD19+ cells, and are all are negative for binding to cells that do not express CD19.

The antibodies described herein bind CD19-positive Burkitt's lymphoma cell lines Daudi (ATCC® CCL-2131M), Raji (ATCC® CCL-86™), and Ramos (ATCC® CRL-1596™), and some are cross-reactive with the CD19 protein of Cynomolgus macaque. In addition, they can be engineered to provide cross-reactivity with the CD19 protein of any animal species, if desired.

The anti-CD19 heavy chain antibodies, such as UniAbs™ herein may have an affinity for CD19 with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g., blocking, a CD19 biological activity, including in vitro assays, pre-clinical models, and clinical trials, as well as assessment of potential toxicity.

Heavy chain antibodies binding to non-overlapping epitopes on a CD19 protein, e.g., UniAbs™ can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details see the examples herein.

Typically, an antibody “competes” with a reference antibody if it causes about 15-100% reduction in the binding of the reference antibody to the target antigen, as determined by standard techniques, such as by the competition binding assays described above. In various aspects, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Pharmaceutical Compositions

It is another aspect of the present disclosure to provide pharmaceutical compositions comprising one or more multispecific binding compounds of the present disclosure in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one aspect, a pharmaceutical composition comprises a heavy chain antibody (e.g., UniAb™) that binds to CD19. In another aspect, a pharmaceutical composition comprises a multi-specific (including bispecific) heavy chain antibody (e.g., UniAb™) with binding specificity for two or more non-overlapping epitopes on a CD19 protein. In a preferred aspect, a pharmaceutical composition comprises a multi-specific (including bispecific) heavy chain antibody (e.g., UniAb™) with binding specificity to CD19 and with binding specificity to a binding target on an effector cell (e.g., a binding target on a T cell, such as, e.g., a CD3 protein on a T cell).

Pharmaceutical compositions of the antibodies used in accordance with the present disclosure are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The antibodies herein can be administered by intravenous injection or infusion or subcutaneously. For injection administration, the antibodies herein can be formulated in aqueous solutions, preferably in physiologically-compatible buffers to reduce discomfort at the site of injection. The solution can contain carriers, excipients, or stabilizers as discussed above. Alternatively, antibodies can be in lyophilized form for constitution with a suitable vehicle, e.g., sterile pyrogen-free water, before use.

Antibody formulations are disclosed, for example, in U.S. Pat. No. 9,034,324. Similar formulations can be used for the heavy chain antibodies, including UniAbs™, of the present disclosure. Subcutaneous antibody formulations are described, for example, in US20160355591 and US20160166689.

### IV. Methods of Use

The antibodies and pharmaceutical compositions described herein can be used for the treatment of diseases and conditions characterized by the expression of a target protein (e.g., CD3, CD19), including, without limitation, the conditions and diseases described further herein. In preferred aspects, the antibodies and pharmaceutical compositions described herein can be used for the treatment of diseases and conditions characterized by the expression of CD19.

The pharmaceutical compositions herein comprising anti-CD19 antibodies can be used to treat disorders characterized by the expression of CD19, including, without limitation, non-Hodgkin lymphoma.

Effective doses of the compositions of the present disclosure for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other agent (e.g., another ablative agent) dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the present disclosure are kits comprising the active agents and formulations thereof, of the disclosure and instructions for use. The kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

Aspects of the disclosure include methods for evaluating the safety, clinical pharmacokinetic (PK), and clinical activity of TNB-486 in subjects with B-NHL. As noted above, B-NHL subtypes can include, but are not limited to, chronic lymphocytic leukemia/small lymphocytic lymphoma, follicular lymphoma (FL), marginal zone lymphoma (MZL), diffuse large B-cell lymphoma (DLBCL), high grade B-cell lymphoma (HGBL), and mantle-cell lymphoma (MCL). For purposes of the current study, B-NHL subtypes include follicular lymphoma (FL), marginal zone lymphoma (MZL), diffuse large B-cell lymphoma (DLBCL), high grade B-cell lymphoma (HGBL), and mantle-cell lymphoma (MCL).

In some aspects, a patient has received at least 2 lines of systemic therapy approved for B-NHL. In some aspects, a patient has previously been exposed to, intolerant of, ineligible for, or declined therapy to a typical second line therapy such as high-dose chemotherapy followed by autologous (Stem Cell Transplant (SCT).

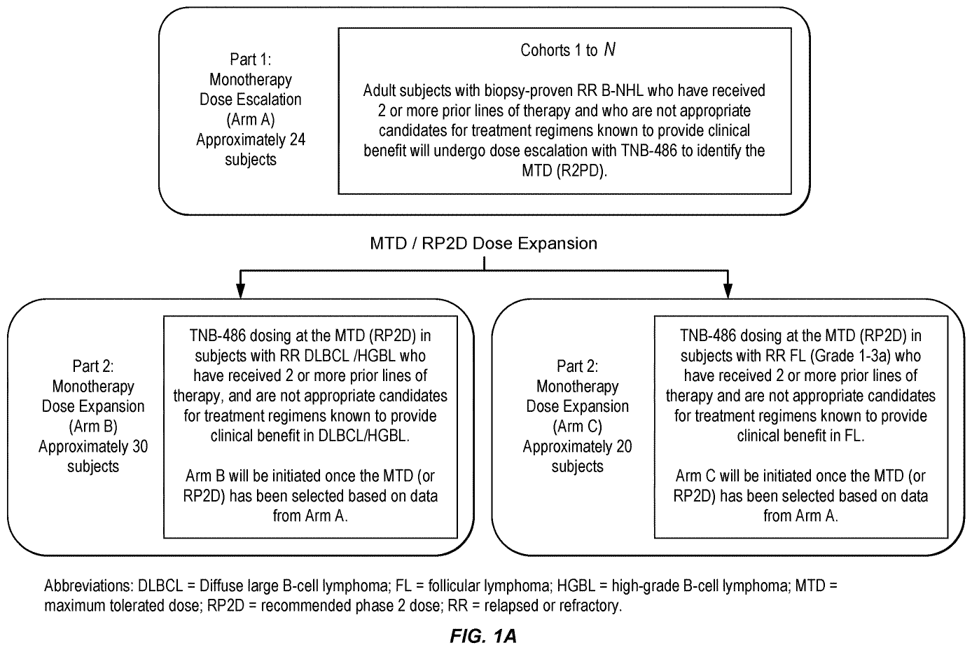

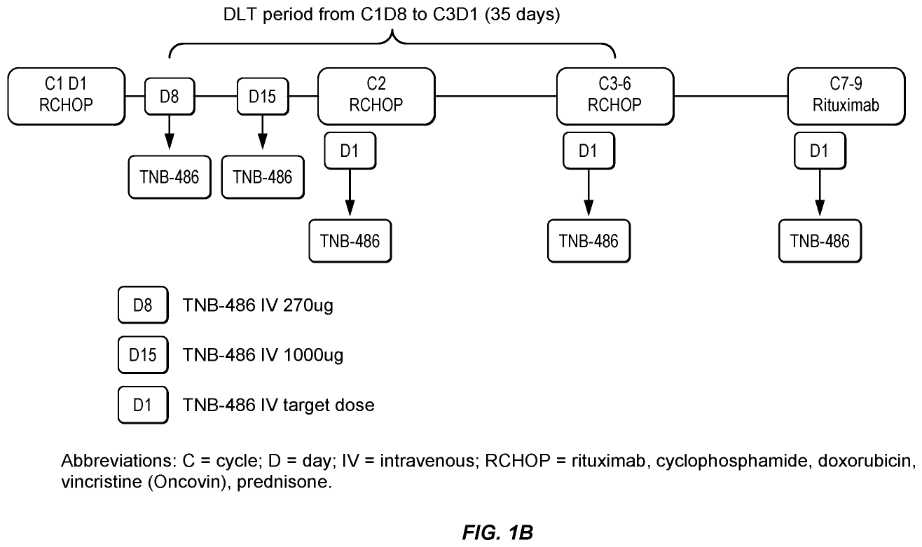

Aspects of the disclosure include methods that involve a Monotherapy Dose Escalation (Part 1, Arm A) and a Monotherapy Dose Expansion (Part 2, Arm B and Arm C). A study design schematic for this administration is presented in FIG. 1A. Some aspects include methods that involve TNB-486 Dose Escalation in Combination with R-CHOP (Part 3, Arm D). An administration schematic for the combination dosing regime is presented in FIG. 1B.

Aspects of the disclosure include methods of administration where dose limiting toxicities (DLTs) occur at a lower rate that other methods of administration. In some aspects, a DLT that occurs is cytokine release syndrome (CRS). CRS and its grades of severity are described herein. In some aspects, the patient does not experience CRS or only experiences Grade 1 CRS. In some aspects, the patient does not experience CRS. In some aspects, the patient does not experience CRS or only experiences Grade 1 or Grade 2 CRS. In some aspects, any CRS experienced by the patient is resolved without treatment of the CRS. In some aspects, any CRS experienced by the patient is resolved with treatment of the CRS. In aspects, the CRS is treated by administration of Tocilizumab.

In some aspects of the methods, the patient has a less than 20% chance of experiencing a Grade 2 or Grade 3 CRS. In aspects, the patient has a less than 24% chance of experiencing a Grade 2 or Grade 3 CRS. In aspects, the patient has a 40% chance of not experiencing CRS.

In aspects, the patient only experiences CRS during the first administration cycle. In aspects, the patient has an about 53% or less chance of experiencing CRS on day 1 of the first administration cycle. In aspects, the patient has an about 15% or less chance of experiencing CRS on day 15 of the first administration cycle. In aspects, the patient does not experience CRS during the second administration cycle. In aspects, the patient does not experience CRS during the second administration cycle or any subsequent administration cycle.

### Monotherapy Dose Escalation (Part 1, Arm A):

In some aspects, the methods involve evaluating the safety, tolerability, PK and PD profiles of single-agent TNB-486 therapy administered once every four weeks (Q2W, 28-day cycle), in patients with B-NHL who have received at least 2 prior lines of therapy. In some aspects, patients have previously been exposed to, intolerant of, ineligible for, or declined therapy to a typical second line therapy such as high-dose chemotherapy followed by autologous (Stem Cell Transplant (SCT). In Arm A, cohorts will initially enroll single subjects.

In some aspects, the methods involve administering a single dose of TNB-486 on a 28-day cycle for at least one cycle. In some aspects, a TNB-486 dose is selected from the group consisting of: 30 μg, 90 μg, 270 μg, 800 μg, 2400 μg, 7200 μg, 15000 μg, and 30000 μg. In some aspects, the methods involve modifying the frequency of dosing to include additional time between doses. In some aspects, the treatment cycle is repeated two or more times. In some aspects, TNB-486 is administered to the patient as a monotherapy. In some aspects, TNB-486 is administered by an intravenous infusion (IV).

In some aspects, the patient will receive one or more priming doses prior to the target dose. In some aspects, the patient will receive a single priming dose on day 1 of the first administration cycle. In some aspects, the patient will receive two priming doses. In some aspects, the patient will receive two priming doses, with the first priming dose administered on day 1 of the first administration cycle and the second priming dose administered on day 2 of the first administration cycle.

In some aspects, Arm A involves a dose escalation design to evaluate the safety, tolerability, PK and PD profiles of single-agent TNB-486 administered Q2W, in up to 24 patients with B-NHL who have received at least 2 prior lines of therapy. In some aspects, patients have previously been exposed to, intolerant of, ineligible for, declined therapy, or are not appropriate candidates for treatment regimens known to provide clinical benefit in B-NHL. In Arm A, cohorts initially enroll single subjects. In some aspects, nine dose levels are evaluated for TNB-486 (Table), but the number of dose levels tested depends on safety, PK/PD and activity data.

In Arm A, cohorts will initially enroll single subjects (FIG. 2 and Table 5). Eight dose levels are proposed for TNB-486 (Table 5), but the number of dose levels tested will depend on tolerability. In some aspects, a dose level is selected from the following: 30 μg, 90 μg, 270 μg, 800 μg, 2400 μg, 7200 μg, 15000 μg, 30000 μg, 40000 μg, 50000 μg, 60000 μg, 70000 μg, 80000 μg, 90000 μg and 100000 μg.

In aspects where at least one priming dose is administered, the priming dose is from 150 μg to 1500 μg. In aspects, the priming dose is from 175 μg to 1250 μg. In aspects, the priming dose is from 200 μg to 1000 μg. In aspects the priming dose is from In aspects, the priming dose is from 200 μg to 500 μg. In aspects, the priming dose is from 250 μg to 1000 μg. In aspects the priming dose is from In aspects, the priming dose is from 250 μg to 500 μg. In aspects the priming dose is from In aspects, the priming dose is from 270 μg to 1000 μg.

In aspects of the methods, two priming doses are administered. In aspects where two priming doses are administered, the first priming dose is from about 150 μg to about 540 μg. In aspects, the first priming dose is from about 175 μg to about 500 μg. In aspects, the first priming dose is from about 200 μg to about 400 μg. In aspects, the first priming dose is from about 200 μg to about 300 μg. In aspects, the first priming dose is from about 250 μg to about 300 μg. In aspects, the first priming dose is about 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, or 350 μg. In aspects, the first priming dose is about 270 μg. In aspects, the first priming dose is 270 μg.

In aspects where two priming doses are administered, the second priming dose is from about 800 μg to about 1200 μg. In aspects, the second priming dose is from about 850 μg to about 1150 μg. In aspects, the second priming dose is from about 900 μg to about 1100 μg. In aspects, the second priming dose is from about 950 μg to about 1050 μg. In aspects, the second priming dose is about 950 μg, 960 μg, 970 μg, 980 μg, 990 μg, 1000 μg, 1010 μg, 1020 μg, 1030 μg, 1040 μg, or 1050 μg. In aspects, the second priming dose is about 1000 μg. In aspects, the second priming dose is 1000 μg.

In some aspects, two priming doses are administered and the first priming dose is about 270 μg and the second priming dose is about 1000 μg.

TABLE 5
Part 1: Monotherapy Dose Escalation - Arm A

| | Priming | Priming | Target | Approx. # of |
|---|---|---|---|---|
| | Dose^{b} | Dose | Dose | Subjects/ |
| Cohorts^{a} | (μg) - step 1 | (μg) - step 2 | (μg) | Cohort^{d} |
| 1 | NA | NA | 30 | 1 |
| 2 | NA | NA | 90 | 1 |
| 3 | NA | NA | 270 | 1 |
| 4a | NA | NA | 800 | 3 |
| 4b | 270 | NA | 800 | 7 |
| 5a | NA | NA | 2400 | 6 |
| 5b | 270 | NA | 2400 | 7 |
| 5d^{c} | 270 | 1000 | 2400 | 7 |
| 6a | 1000 | NA | 7200 | 6 |
| 6c^{c} | 270 | | 7200 | 1 |
| 6d^{e} | 270 | 1000 | 7200 | 7 |
| 7a | 1000 | NA | 10000 | 7 |
| 7d^{e} | 270 | 1000 | 10000 | 7 |
| 8d | 270 | 1000 | 15000 | 7 |

Abbreviations: DLT = dose-limiting toxicity; MTD = maximum tolerated dose; N = cohort number; RP2D = recommended phase 2 dose; SMG = Safety Monitoring Group.
^{a}Dose de-escalation from any dose level (except starting 30 μg) may occur to refine the determination of the MTD and/or RP2D.
^{b}Single priming doses will be explored in cohorts 4b, 5b, 6a and 7a. Beginning with Cohort 4b, cohorts Na, b, c, etc. may be conducted in parallel. Not all dose level cohorts will be enrolled based on emerging safety data.
^{c}Cohort 6c is closed for enrollment
^{d}The approximate number of subjects is based on lack of dose associated toxicities in any cohort. The actual numbers of subjects will depend on safety and other findings. No more than 3 subjects will be enrolled concurrently in a given sub-cohort.
^{e}Cohorts 5d, 6d and 7d are not part of the dose-escalation design but considered backfill of cohort 5b, 6a and 7a, respectively. No formal DLT assessment is planned for cohorts that are considered backfill. Once Cohort 7a is considered safe for escalation by SMG as guided by BOIN, enrollment into Cohort 8d may start.

Upon the first occurrence of a Grade ≥2 adverse event (AE) that is not unequivocally due to the subject's underlying malignancy or other extraneous cause, the corresponding cohort, and all subsequent cohorts in Arm A, will be expanded to 3 subjects and will follow a standard 3+3 dose escalation design. If a DLT occurs in a single subject cohort, the cohort will be expanded to 6 subjects. If a second DLT occurs in a cohort, the immediately preceding dose is the MTD. If only 1 DLT is observed, subsequent cohorts will proceed according to a 3+3 dose escalation design.

If a response (PR or CR) is observed in a single subject cohort, cohorts enrolling thereafter will proceed according to a 3+3 dose escalation design.

TABLE 6
Part 1: Monotherapy Dose Escalation Arm A Guidelines

| Number of Subjects | |
|---|---|
| with DLT | Dose Escalation Outcomes |
| 0 of 1 | Begin enrollment in the next dose level. |
| 1 of 1 (≥Grade 2+) | Enroll a total of 3 subjects in current dose level. |
| 1 of 1 (DLT) | Enroll a total of 6 subjects in current dose level. |
| 0 of 3 | Begin enrollment in the next dose level. |
| 1 of 3 | Enroll a total of 6 subjects in current dose level. |
| 1 of 6 | Begin enrollment in the next dose level. |
| 2 of 6 (or ≥33.3%) | Dose escalation stops and previous dose |
| | determined as MTD or new intermediate |
| | dose evaluated. |

Abbreviations: DLT = dose limiting toxicity; MTD = maximum tolerated dose.

In some aspects, dose escalation will begin with a Q2W dosing schedule, which may be altered after cumulative review of safety and PK data and a protocol amendment. Administration of the first dose of TNB-486 to the first subject in each cohort must await completion of the first cycle (4 weeks) of the prior dose level, and a review of the safety data by the Medical Monitors (Syneos, Inc. and TeneoTwo), and the Principal Investigators (or designated sub investigators; hereafter referred to as the SMG. Input from biostatistical, PK and other experts will be sought as necessary.

In some aspects, based on the SMG's ongoing review of emerging safety and clinical pharmacology data, cohorts receiving intermediate doses between those proposed above may be implemented. Furthermore, it may become necessary to switch to an alternative dosing regimen. For example, dosing may be switched to a different frequency (e.g., once every 3 weeks) or some cycles may be consistently eliminated from a dosing schedule (e.g., every scheduled 3rd cycle may be dropped). If the dosing schedule is switched to occur more frequently, no dose modification should result in a predicted steady state concentration (CSS) or Cmax greater than those identified for the next lower dose level. If new intermediate dose levels and/or a new dosing schedule is/are to be evaluated, a protocol amendment with appropriate justification will be submitted.

### Dose Escalation and Selection of the MTD/RP2D Dose:

In some aspects, DLT criteria are used to make decisions regarding dose escalation. Dose escalation decisions will be made by the SMG based on clinically significant toxicity, DLT events, and PK and PD findings (when available). A simple majority of the SMG is required to proceed with dose escalation.

Dose escalation will proceed as follows (and as illustrated in FIG. 2):
In single subject cohorts, if the first DLT evaluable subject in a given cohort completes the safety assessment during the first cycle without evidence of activity and without experiencing a Grade ≥2 AE not unequivocally due to their underlying malignancy or other extraneous cause, escalation to the next proposed dose level will proceed.
If a subject in a single subject cohort experiences a non-DLT Grade ≥2 AE not unequivocally due to their underlying malignancy or other extraneous cause, that same dose level will be expanded to 3 subjects.
If the first 3 DLT evaluable subjects in a 3 subject cohort complete the safety assessment during the first cycle without experiencing a DLT, escalation to the next proposed dose level will proceed.
If 1 subject at a dose level experiences a DLT (including single subject cohorts), that same dose level will be expanded to 6 subjects.
If 1 of 6 evaluable subjects experience a DLT, escalation to the next proposed dose level will proceed.
If 2 of 6 subjects experience a DLT, dose escalation will stop because the MTD will have been exceeded.

In some aspects, the SMG safety review of cycle 1 for a cohort Nis complete and if that dose is deemed safe (e.g. the SMG endorses further dose escalation or the dose for cohort N is determined to be the RP2D), any subjects that remain on study at a lower dose of TNB-486 may subsequently be treated at the dose assigned to cohort N (e.g. when the SMG review for cohort 5 is complete and the decision to escalate to cohort 6 has been made, any subjects still on study from cohorts 1-4 may have their dose increased to the dose corresponding to cohort 5). Subject eligibility for such an escalation must be approved by the SMG and will be determined on a case-by-case basis. At a minimum, subjects must have previously received at least 2 cycles of TNB-486 at their current dose and had at least 1 post-dose disease assessment, without any drug-related toxicities leading to a dose reduction, to be eligible. Subjects should have Stable Disease or better and should not have experienced any grade ≥2 AE that is not unequivocally due to underlying disease or other extraneous cause with their most recent dose of TNB-486.

In some aspects, if the available data during SMG review of cohort N are equivocal regarding further escalation (e.g. based on occurrence of non-DLT AEs, plateaued efficacy, suspected RP2D), cohort N and/or cohort N−1 may be expanded up to a maximum of 9 subjects each at the discretion of the SMG.

### Dose Priming:

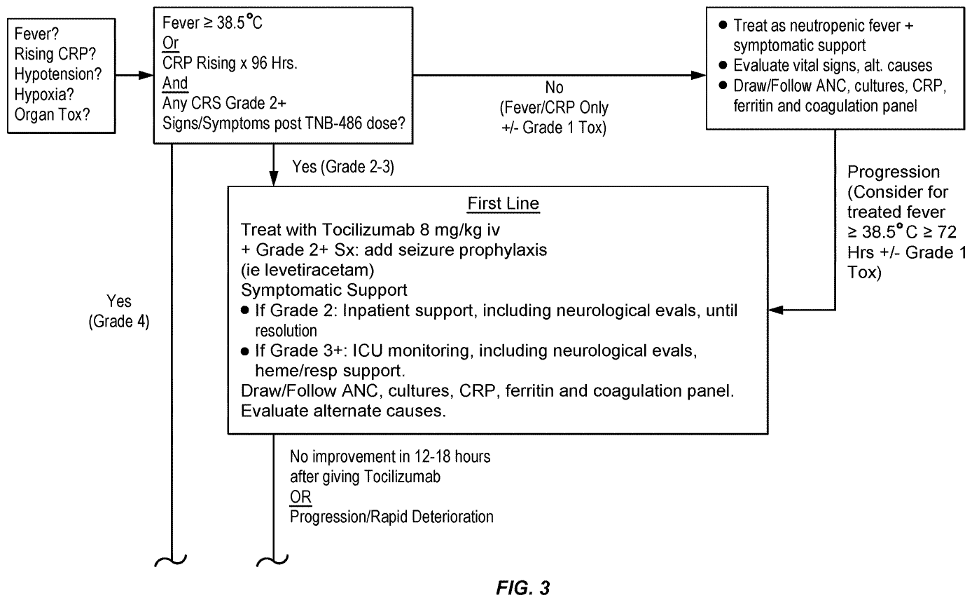

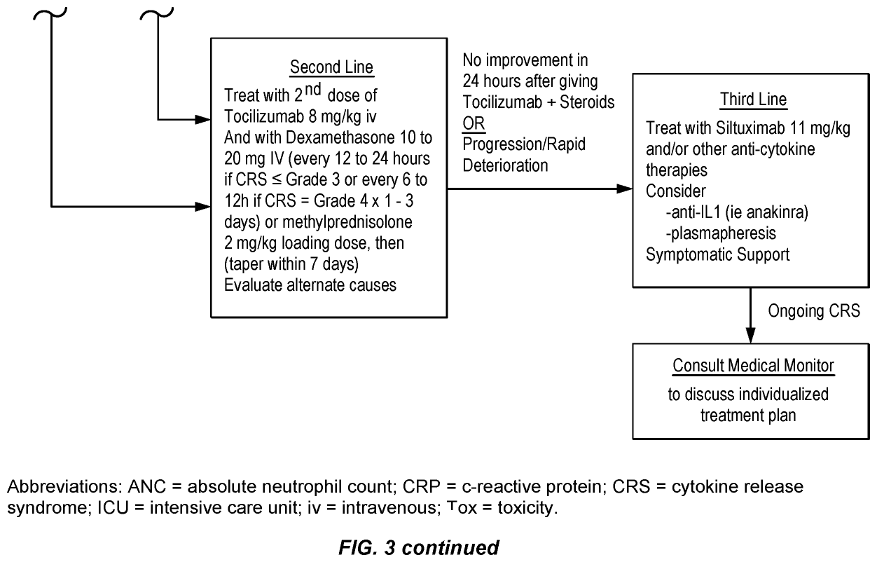

Based on the non-clinical pharmacology of TNB-486, a lower rate of CRS is anticipated than with other T-cell-engaging bispecifics; however, based on the mechanism of action of TNB-486, the most likely DLT to occur is still CRS ≥Grade 3. Cytokine release syndrome is to be managed per Principal Investigator discretion and institutional guidelines (a guide for management of CRS is provided in FIG. 3 if institutional guidelines have not been established).

In some aspects, if a Grade ≥3 CRS event occurs in Cohort N during the DLT period, or at the discretion of the SMG, a dose equal to or less than the dose for Cohort N is to be designated as the ‘Priming Dose’ and dose administration in subsequent cohorts (Cohorts N+1 and on) will be modified as follows:
On Cycle 1 Day 1, subjects will receive the Priming Dose. The priming dose in cohorts N+1 and higher will not exceed the dose administered in cohort N.
In aspects where a Second Priming Dose is administered, on Cycle 1, Day 8, subjects will receive the Second Priming Dose.
From Cycle 1 Day 15 on subjects will receive the full dose corresponding to the cohort in which they have been enrolled.
In cohort N+1 the full dose will not exceed the priming dose (i.e. the dose administered to cohort N) by more than 50%.
The full dose administered in cohorts N+2 and higher should not exceed+100% of the full dose in the immediately preceding cohort.
If 2 grade ≥3 CRS AEs occur in the same cohort dose escalations of the full dose should not exceed+50% of the immediately preceding cohort thereafter.
If a DLT occurs, dose escalations of the full dose should not exceed+33% of the immediately preceding cohort thereafter.

After dosing on Cycle 1 Day 1, if a subject experiences any Grade ≥3 toxicity that is not unequivocally due to the subject's underlying malignancy or other extraneous cause, administration of the full dose on Cycle 1 Day 15 must await approval by the Medical Monitors. At a minimum, CRS must have resolved to Grade ≤1 before the full dose is administered. The dose and timing of the full dose for such a subject may be modified at the discretion of the Medical Monitors and the Investigator; if the subject has their full dose reduced in cycle 1, the subject will be considered DLT-unevaluable and should be replaced, unless they experienced a DLT. In subsequent cycles, subjects will receive the full dose on Days 1 and 15.

Following implementation of a priming dose, if administration of the full dose is delayed in Cycle 1, the DLT period for that subject should be extended to the same degree (e.g. if the full dose is delayed by 4 days, the DLT period will also be extended 4 days). For clarity, treatment-emergent adverse events (TEAEs) meeting criteria for a DLT will be treated as a DLT whether they occur after administration of the priming or the full dose.

### Monotherapy Dose Expansion (Part 2, Arm B):

In some aspects, the methods involve evaluating an MTD (or RP2D) of TNB-486 monotherapy in approximately 30 subjects with biopsy-proven RR DLBCL or HGBL. To be eligible for this study, subjects must have received 2 or more prior lines of therapy, and must not be appropriate candidates for treatment regimens known to provide clinical benefit in DLBCL/HGBL. At least 15 subjects should not have previously been treated with a CD19 targeted therapy. Arm B will be initiated once the MTD (or RP2D) has been selected based on data from the Monotherapy Dose Escalation (Part 1, Arm A). The MTD (or RP2D) and dosing frequency for Arm B will be chosen by the SMG based on safety, tolerability, and PK/PD data collected during the dose escalation portion of the study.

### Monotherapy Dose Expansion (Part 2, Arm C):

In some aspects, the methods involve evaluating an MTD (or RP2D) of TNB-486 monotherapy in approximately 20 subjects with biopsy-proven RR FL (Grade 1-3a). To be eligible for this study, subjects must have received 2 or more prior lines of therapy and must not be appropriate candidates for treatment regimens known to provide clinical benefit in FL. At least 10 subjects should not have previously been treated with a CD19 targeted therapy. Arm C will be initiated once the MTD (or RP2D) has been selected based on data from the Monotherapy Dose Escalation (Part 1, Arm A). The MTD (or RP2D) and dosing frequency for Arm C will be chosen by the SMG based on safety, tolerability, and PK/PD data collected during the dose escalation portion of the study.

TNB-486 Dose Escalation in Combination with R-CHOP (Part 3, Arm D):

In some aspects, the methods involve evaluating the safety, tolerability, PK, and pharmacodynamic profiles of TNB-486 in combination with R-CHOP administered every 3 weeks in approximately 9 to 18 subjects. A schematic study design for Part 3 Arm D is presented in FIG. 1B.

All subjects will receive six cycles of TNB-486 plus R-CHOP chemotherapy at 21-day intervals (Cycles 1-6), followed by three additional cycles of TNB-486 and rituximab (Cycles 7-9) for a total of 9 treatment cycles. R-CHOP will be administered as per standard of care with fixed doses. In Cycle 1, all subjects will receive R-CHOP on Day 1 followed by two escalating step-up doses for TNB-486 on Days 8 and 15. On Cycle 2 Day 1, RCHOP plus TNB-486 will be administered at target dose IV Q3W. Dose escalation will start with Dose Level (DL) 1 (i.e. TNB-486 270 ug Cycle 1 Day 8, followed by 1000 ug on Cycle 1 Day 15; R-CHOP plus TNB-486 7200 μg on Cycle 2 Day 1). Based on DLT distribution, subsequent dose levels will be de-escalated or escalated into DL-1 or DL 2, respectively, as per Table 7.

TABLE 7
TNB-486 Dose levels tested in combination with R-CHOP

| | | | Target | Approx. |
|---|---|---|---|---|
| | Priming Dose | Priming Dose | Dose | Number of |
| Cohorts | (μg) - Step 1 | (μg) - Step 2 | (μg) | Subjects/Cohort |
| Dose Level 1 | 270 | 1000 | 2400 | 3-6 |
| Dose Level 1 | 270 | 1000 | 7200 | 3-6 |
| Dose Level 2 | 270 | 1000 | 10000 | 3-6 |

Abbreviation: R-CHOP = rituximab, cyclophosphamide, doxorubicin, vincristine (Oncovin), prednisone.

As common treatment-related toxicities for T-cell engagers (i.e. CRS and NT) typically occur within 24 to 72 hours post infusion, in some aspects, the first subject in each cohort will complete at least 1 week of observation after receiving the target dose of TNB-486 plus R-CHOP on Cycle 2 Day 1, before additional subjects may receive the target dose in that given cohort. In some aspects, the DLT window will be counted from Cycle 1 Day 8 to Cycle 3 Day 1 (total 3 TNB-486 doses, 35 days).

In some aspects, subjects in Part 3, Arm D will be enrolled according to an interval 3+3 (i3+3) design with the target toxicity rate of 30% and equivalence interval (EI) of (25% to 35%). In aspects, up to 3 subjects may be enrolled concurrently. In aspects, the de-escalation or escalation decision will be made based on a minimum of 3 DLT-evaluable subjects for a cohort. In aspects, approximately 9 to 18 subjects will be enrolled, assuming 3 dose levels will be investigated, and 3 to 6 subjects will be assigned at each dose level. In aspects, the total number of subjects will depend upon the number of dose escalations/de-escalations necessary.

In aspects, once active dose cohorts of TNB-486 in combination with R-CHOP are reached (i.e. cohorts in which at least one partial response or better is observed) and if the DLT rate for the highest dose cohort inside the EI (i.e., not mandating dose reduction) at the time of an SMG meeting, active dose cohorts <N may be expanded up to a maximum of 15 subjects each at the discretion of the SMG to better assess the optimal biologic dose/RP2D. In aspects, if one or more dose level is expanded, up to a maximum of 15 subjects will be allocated within the cohorts, respectively.

In aspects, if a DLT attributed to TNB-486 per the investigator's assessment occurs, dosing will be withheld. TNB-486 treatment can be resumed at a lower dose only after toxicity has resolved to Grade ≤1 if, in the opinion of the treating Investigator, the patient may derive clinical benefit. In aspects, there will be no dose re-escalation for TNB-486 after recovery from toxicity, and no intra-cohort participant dose escalation will be allowed. In aspects, dose adjustments for R-CHOP components will follow conventional dose modification schedules.

### Selection of Study Population:

In some aspects, patients undergo screening procedures within 28 days prior to initial study drug administration. Adult patients who meet the inclusion criteria and who do not meet any of the exclusion criteria will be eligible for enrollment into the study.

### Inclusion Criteria:

1. Subject must be ≥18 years of age.
2. Subject has biopsy proven B-NHL per World Health Organization (WHO) Criteria (Swerdlow 2017).
3. For Arm B Only: Subject has biopsy proven RR DLBCL or HGBL per WHO Criteria (Swerdlow 2017). At least 15 subjects in Arm B should not have previously been treated with a CD19 targeted therapy.
4. For Arm C Only: At least 10 subjects in Arm C should not have previously been treated with a CD19 targeted therapy.
5. For Arm D Only: Subject has biopsy proven, previously untreated, CD20-positive DLBCL, including one of the following diagnoses per WHO Criteria (Swerdlow 2017):
   a. DLBCL, not otherwise specified (NOS)
   b. T-cell/histiocyte-rich large B-cell lymphoma
   c. Epstein-Barr virus-positive DLBCL, NOS
   d. ALK-positive large B-cell lymphoma
   e. High-grade B-cell lymphoma with MYC and BCL2 and/or BCL6 rearrangements (double-hit or triple-hit lymphoma)
   f. High-grade B-cell lymphoma, NOS
   g. DLBCL transformed from indolent lymphoma
   h. Primary cutaneous DLBCL, leg 1 type
   i. Follicular lymphoma Grade 3B.
6. Subject's B-NHL disease is CD19-positive.
7. Subject has ≥1 measurable disease site(s) as defined in the RECIL 2017 classification. (Younes 2017).
8. Subject has received at least 2 lines of therapy to which the subject has been either refractory or has subsequently relapsed. In order to be eligible for this study subjects must not be candidates for treatment regimens known to provide clinical benefit in B-NHL.
9. For Arm D Only: Subject has received no prior treatment for the indication under study (except steroids for lymphoma symptom control) and is eligible to receive chemoimmunotherapy with R-CHOP in the investigator's opinion.
10. For Arm D Only: Subject has an IPI score of 2 to 5.
11. For Arm D Only: Subject has a left ventricular ejection fraction (LVEF) within institutional normal limits as determined by cardiac echocardiogram (ECHO).
12. Subject has an Eastern Cooperative Oncology Group (ECOG) Performance Status of ≤2.
13. Subject must have adequate bone marrow function, defined as: absolute neutrophil count (ANC) ≥1000/mm³; platelets ≥50,000/mm³ (≥100,000/mm³ for Part 3 Arm D); hemoglobin ≥8.0 g/dL. Transfusion and/or growth factor support is permitted prior to assessment, but neutrophils, platelets, and hemoglobin must be stable for at least 72 hours after transfusion and/or growth factor administration prior to Screening for the subject to be eligible. If the subject has received a long half-life growth factor (e.g. pegylated granulocyte-colony stimulating factor [G-CSF]), the corresponding cell count must be stable for at least 7 days for the subject to be eligible.
14. Subject must have an estimated glomerular filtration rate (eGFR) ≥50 mL/min as estimated by the MDRD formula. Note: Per FDA guidance, ‘renal impairment is not likely to alter PK enough to justify dosage adjustment’ for protein therapeutics >69 kDa, specifically including antibodies, such as TNB-486 (FDA 2010).
15. Subject must have total bilirubin <1.5× upper limit of normal (ULN; except if the subject has a known diagnosis of Gilbert's syndrome, in which case bilirubin must be <3×ULN). Aspartate aminotransferase (AST) and alanine aminotransferase (ALT) must be ≤3×ULN (except if the subject has a known diagnosis of Gilbert's syndrome, in which case AST/ALT must be <5×ULN).

### Exclusion Criteria:

A subject will not be eligible for treatment if he/she meets any of the following criteria:
1. Subject has been diagnosed with or treated for another malignancy whose natural history or treatment may interfere with the safety or efficacy assessment of the investigational regimen. Patients with a prior or concurrent malignancy whose natural history or treatment does not have the potential to interfere with the safety or efficacy assessment of the investigational regimen are eligible for this trial.
2. Subject has a history of CNS involvement by their B-NHL. If there is a concern for CNS involvement, an MRI and/or spinal tap should be performed at Screening.
3. Subject has a history of leukemic presentation of their B-NHL, with the exception of MCL or MZL.
4. Subject has history or presence of clinically significant CNS pathology (e.g., epilepsy, seizure, stroke, paresis, aphasia, severe brain injury, dementia, neurodegenerative disorder including Parkinson's disease, dementia, cerebellar disease, organic brain syndrome, psychosis or other severe mental illness).
5. Subject has CNS involvement from active or history of autoimmune disease.
6. Subject experienced Grade ≥3 CRS (based on ASTCT criteria) following prior TCE or CAR T-cell therapy.
7. Subject experienced Grade ≥2 neurotoxicity/ICANS (based on ASTCT criteria) following prior TCE or CAR T-cell therapy.
8. Subject has received another investigational drug within 5×T_{1/2} (of this agent) or within 28 days of enrollment, whichever is shorter.
9. Subject has received a peripheral autologous SCT within 12 weeks, or an allogeneic SCT within 1 year of the first dose of study drug treatment or has received an SCT and requires ongoing immunosuppressive therapy.
10. Subject requires chronic immunosuppressive therapy (including steroids >10 mg prednisone/day), for Part 3, Arm D, a pre-phase with steroids is allowed. At the discretion of the Medical Monitor and the Sponsor, subject may be eligible if immunosuppressive therapy is discontinued 14 days or 5 half-lives prior to the first dose of study treatment (whichever is shorter).
11. Subject has any medical or psychiatric condition which, in the opinion of the investigator or Medical Monitor, places the subject at an unacceptably high risk for toxicities, could interfere with successful or safe delivery of therapy, or could interfere with evaluation of the study drug or interpretation of subject safety or study results. Examples include history of significant mucosal/internal bleeding, major psychiatric illness, drug abuse (including active alcoholism), active graft versus host disease, or known allergy or hypersensitivity to components of the study drug formulation.
12. Subject has received any therapy to treat cancer (including radiation, chemotherapy, biologics, cellular therapies) or undergone a major surgical procedure within 14 days (or within 5 half-lives of an anticancer drug) prior to the first dose of study treatment, whichever is shorter. The administration of debulking chemotherapy outside of this washout period to reduce the risk of TLS or CRS/NT in subjects with bulky disease is permitted.
13. Subject has known active infection requiring parenteral antibiotic treatment. Upon completion of parenteral antibiotics and resolution of symptoms, the subject is considered eligible for the study from an infection standpoint.
14. Subjects with human immunodeficiency virus (HIV) infection, or subjects with chronic or active infection with hepatitis B virus (HBV) or hepatitis C virus (HCV). HIV-infected patients on effective anti-retroviral therapy with undetectable viral load within 6 months are eligible for this trial. Subjects with chronic HBV may be enrolled if the HBV viral load is undetectable on suppressive therapy, or if the subject has a documented cure (hepatitis B surface antigen [HBsAg] negative). Subjects with HCV who have a documented cure (undetectable HCV RNA 24 weeks after the end of treatment) may be enrolled.
15. Major cardiac abnormalities, such as but not limited to the following: uncontrolled angina or unstable life-threatening arrhythmias, history of myocardial infarction ≤12 weeks before Screening, Class ≥3 New York Heart Association congestive heart failure, severe cardiac insufficiency, or persistent QTc prolongation (>480 msec, QTc Fridericia).
16. If female, subject must not be pregnant or breastfeeding and be either postmenopausal (for at least 12 consecutive months), OR permanently surgically sterile, OR for women of childbearing potential practicing a protocol specified highly effective method of female birth control (Section 0) starting at Cycle 1 Day 1 through at least 6 months after the last dose of study drug.
17. Subject has unresolved AEs ≥Grade 2 (National Cancer Institute [NCI] Common Terminology Criteria for Adverse Events [CTCAE]v5.0) from prior anticancer therapy except for:
   Alopecia.
   Peripheral neuropathy (peripheral neuropathy ≥Grade 3 will be excluded).
   Anemia or thrombocytopenia (thrombocytopenia must be Grade 4 to trigger exclusion, Grade 3 with symptoms or bleeding, or return within 72 hours despite transfusion support).
   Subjects with irreversible toxicity not reasonably expected to be exacerbated by the study drugs (e.g. hearing loss) may be included after consultation with the Medical Monitor.
18. For Arm D Only: Current diagnosis of any of the following:
   a. B-cell lymphoma unclassifiable, with features intermediate between DLBCL and classical Hodgkin lymphoma (grey-zone lymphoma)
   b. Primary mediastinal (thymic) large B-cell lymphoma
   c. Burkitt lymphoma
   d. HHV8-positive DLBCL, NOS
   e. Primary effusion DLBCL
19. For Arm D: Contraindication to any of the individual components of R-CHOP including prior receipt of anthracyclines.
Contraception Recommendations and Pregnancy Testing: If female, subject must be either postmenopausal defined as:
   Age ≥55 years with no menses for 12 or more consecutive months without an alternative medical cause.
      OR
   Age <55 years with no menses for 12 or more consecutive months without an alternative medical cause AND a follicle stimulating hormone level >40 I/LL.
      OR
   Permanently surgically sterile (bilateral oophorectomy, bilateral salpingectomy or hysterectomy).
      OR
   For women of childbearing potential, practicing a highly effective method of birth control from the following list effective on Cycle 1 Day 1 (or earlier) through at least 6 months after the last dose of study drug:
      Combined (estrogen and progesterone containing) hormonal contraception (oral, intravaginal, transdermal) associated with the inhibition of ovulation, initiated at least 1 month prior to Cycle 1 Day 1.
      Progesterone-only hormonal contraception (oral, injectable, implantable) associated with inhibition of ovulation, initiated at least 1 month prior to Cycle 1 Day 1.
      Bilateral tubal occlusion/ligation.
      Vasectomized partner(s), provided the vasectomized partner has received medical assessment of surgical success and is the sole sexual partner of the trial participant who is a woman of child bearing potential.
      Condoms
      Intrauterine device.
      Intrauterine hormone-releasing system.
      True abstinence: Refraining from heterosexual intercourse when this is in line with the preferred and usual lifestyle of the subject (periodic abstinence [e.g., calendar, ovulation, symptothermal, post-ovulation methods] and withdrawal are not acceptable).
         If male, subjects of reproductive potential (i.e., male subjects who have not undergone a bilateral vasectomy) must agree to use at least 1 male (vasectomy or condoms) highly effective method of contraception during sexual contact with females of childbearing potential starting with the first dose of study medication through at least 6 months after the last dose of study therapy.

### Treatment Administration:

In some aspects, TNB-486 is initially administered as an IV infusion Q2W, where 1 cycle of treatment will be 28 days. A minimum of 1 and up to 9 subjects will be enrolled at each dose level in Arm A. A 30 μg starting dose of TNB-486 will be administered as an IV infusion (Q2W) in the Monotherapy Dose Escalation (Part 1, Arm A) and escalate to a projected maximum of 30000 μg in subsequent cohorts (Table 5). In Part 2, Arms B and C, all subjects will receive TNB-486 at the MTD and/or RP2D. Subjects may continue to receive TNB-486 until they meet criteria for subject discontinuation.

In some aspects, subjects will be premedicated with dexamethasone (10 mg IV) or equivalent. If a subject does not experience an infusion related reaction (IRR) or an immune mediated toxicity (IMT; e.g. CRS or NT) in a given Cycle, the dexamethasone premedication dose may be reduced to 5 mg IV. If a subject does not experience an IRR or IMT in a cycle where they received 5 mg dexamethasone IV as premedication, dexamethasone may subsequently be omitted from the premedication regimen. If a subject experiences an IRR or IMT at any time, or if the subject undergoes an intra-subject dose escalation, they should be premedicated with 10 mg dexamethasone with the next dose of TNB-486 and tapered as described above.

In some aspects, subjects are routinely premedicated with diphenhydramine (25 to 50 mg IV) or equivalent (e.g., cetirizine 10 mg orally [PO]×1), acetaminophen 650 to 1000 mg PO, and ranitidine 150 mg PO/IV or equivalent, 15 to 60 minutes prior to TNB-486 infusion to reduce the risk and severity of hypersensitivity reactions commonly observed with mAb therapy. Subjects may also be premedicated with tocilizumab (8 mg/kg IV) at the discretion of the investigator and after approval by the Medical Monitors.

In some aspects, the first TNB-486 infusion is given over 2 hours (±10 minutes). The duration of infusion may be shortened after selection of the RP2D. Subjects will be admitted for 48 hours as inpatients after the infusion on Day 1 of Cycle 1; if a subject received a priming dose on C1D1 or experienced CRS Grade ≥2 with their first dose they will be admitted for 48 hours on C1D15 as well. Subjects will be monitored closely for 2 hours after each infusion in subsequent cycles. Please see the Pharmacy Manual for additional details on dose modifications.

In some aspects, the dose(s) selected for evaluation in the Monotherapy Dose Expansion (Part 2, Arms B and/or C) may be a dose at or below the MTD defined in the Monotherapy Dose Escalation (Part 1, Arm A) or the RP2D. Dosing frequency for the Monotherapy Dose Expansion (Part 2, Arms B and/or C) will be chosen by the Sponsor based on safety, tolerability, and PK/PD data collected from the Monotherapy Dose Escalation (Part 1, Arm A), in consultation with the SMG.

### Preparation Reconstitution of Dosage Form:

In some aspects, the TNB-486 drug product (active) is provided as a solution in vials, formulated at 2 mg/mL with 8 mL of extractable volume of drug product per vial, and administered by IV infusion. For the first 4 cohorts in the Monotherapy Dose Escalation (Part 1, Arm A; Table 5) TNB-486 will be diluted in 2 steps. The first dilution step reduces the strength of TNB-486 100-fold to 20 μg/mL using a non-DEHP 50 mL IV bag, provided with the kit. The second dilution step requires dose-dependent transfer of a specified volume of pre-diluted TNB-486 into a non-DEHP 100 mL IV bag (100 mL non-DEHP-containing IV bags will be provided by TeneoTwo). The final concentrations for the first 4 dose cohorts are 240 ng/mL, 720 ng/mL, 2.16 μg/mL and 6.48 μg/mL, respectively. The diluent for each dilution step is saline with IV stabilizer solution (IVSS) added prior to the addition of active TNB-486 drug product. The IVSS is provided with each kit and consists of a 20 mL glass vial with 15 mL extractable volume. The IVSS vial is formulated at 10× strength, with a working strength of 1× in the IV bag.

In some aspects, study drug for dose cohorts 5 and higher (Table 5) will be prepared in a single dilution step, with dose dependent volumes of TNB-486 transferred from drug product vial directly into a 100 mL non-DEHP IV bag, provided with each kit. The diluent is saline with IVSS added prior to the addition of active TNB-486 drug product, identical in formulation compared to the lower dose cohorts. The concentrations of active drug product range from 24.3 μg/mL to 303.7 μg/mL in dose cohorts 5 and higher.

For all cohorts, total storage time (including infusion time) of the IV bag containing the final dilution of TNB-486 at controlled room temperature (20 to 25° C.) should not exceed 6 hours (or 24 hours at 2 to 8° C.) to minimize degradation of the drug product and the risk of microbiological contamination. The storage time may be updated as additional sterility/stability data become available.

In some aspects, the total volume administered at each dose is 250 mL. Infusion rates are controlled with infusion pumps and their respective DEHP-free infusion sets containing inline filters.

In some aspects, the TNB-486 drug product vials are stored at 5±3° C. The IVSS vial is stored at ambient temperature. The diluted active drug preparations have been tested at the lowest (240 ng/mL) and highest dose (303.7 μg/mL) with exposure to light for infusion set compatibility for up to 6 hours at controlled room temperature (20 to 25° C.) and for up to 24 hours at 2 to 8° C.

### Eastern Cooperative Oncology Group (ECOG) Performance Status:

Aspects of the disclosure include assessing an ECOG performance status of a subject at various time points throughout the study. The ECOG performance status is documented using the scoring method in Table 8.

TABLE 8
Eastern Cooperative Oncology Group (ECOG) Performance Score

| Grade | ECOG |
|---|---|
| 0 | Fully active, able to carry on all pre-disease performance without restriction. |
| 1 | Restricted in physically strenuous activity but ambulatory and able to carry out |
| | work of a light or sedentary nature, e.g., light housework, office work. |
| 2 | Ambulatory and capable of all self-care but unable to carry out any work |
| | activities. Up and about more than 50% of waking hours. |
| 3 | Capable of only limited self-care, confined to bed or chair more than 50% of |
| | waking hours. |
| 4 | Completely disabled. Cannot carry on any self-care. Totally confined to bed or |
| | chair. |

### Pregnancy Tests:

Aspects of the disclosure include obtaining serum or urine to perform a pregnancy test for women of childbearing potential at Screening by a central laboratory, and a serum or urine pregnancy test must be conducted locally with a negative result within 72 hours of dosing. Thereafter, a urine pregnancy test (with confirmation via a serum pregnancy test if positive) must be performed locally and documented negative prior to initiating dosing for every cycle.

### Clinical Laboratory Tests:

Aspects of the disclosure include obtaining samples for the clinical laboratory tests outlined in Table 9 at a minimum at Screening, and locally at subsequent visits, the EOT Visit, and the 90-day follow-up visit as outlined in the Schedule of Events (All Arms).

In some aspects, a certified laboratory is utilized to process and provide results for the clinical laboratory tests. Laboratory reference ranges are obtained prior to the initiation of the study. The baseline laboratory test results for clinical assessment for a particular test are defined as the last measurement prior to the initial dose of TNB-486.

TABLE 9
Clinical Laboratory Tests

Laboratory

| | |
|---|---|
| Test | Parameter |

| | | |
|---|---|---|
| Hematology | Hematocrit | Basophils (if detected; |
| | Hemoglobin | absolute count and %) |
| | RBC count | Eosinophils (if detected; |
| | WBC count | absolute count and %) |
| | Neutrophils (absolute | Platelet count (estimate |
| | count and %) | not acceptable) |
| | Bands (if detected) | MCH |
| | Lymphocytes (absolute | MCV |
| | count and %) | MCHC |
| | Monocytes (absolute | |
| | count and %) | |
| Coagulation | INR | aPTT |
| Panel | PT | |
| Clinical | BUN | Serum pregnancy test |
| Chemistry | Creatinine | (where applicable) |
| | Total bilirubin | Total protein |
| | Albumin | Glucose |
| | AST | Chloride |
| | ALT | Bicarbonate/CO₂ |
| | Alkaline phosphatase | LDH |
| | Sodium | Magnesium |
| | Potassium | GGT |
| | Calcium | B2-microglobulin |
| Cytokines ^{a} | IL-2 | IFN-gamma |
| | IL-6 | IL-1 |
| | TNF-alpha | IL-10 |
| Viral Testing | HBsAg | |
| (Screening | HCV Ab | |
| only) | HIV | |
| Urine Tests | Urine pregnancy test | Urobilinogen |
| (Screening | Protein | Bilirubin |
| only) | Glucose | Microscopic examination |
| | Blood | |

Abbreviations:
Ab = antibody;
aPTT = activated partial thromboplastin time;
ALT = alanine aminotransferase;
AST = aspartate aminotransferase;
BUN = Blood urea nitrogen;
GGT = gamma-glutamyltransferase;
HBsAg = hepatitis B surface antigen;
HCV = hepatitis C virus;
IFN = interferon;
IL = interleukin;
LDH = lactate dehydrogenase;
MCH = mean corpuscular hemoglobin;
PT = prothrombin time;
MCHC = mean corpuscular hemoglobin concentration;
MCV = mean corpuscular volume;
RBC = red blood cell;
TNF = tumor necrosis factor;
WBC = white blood cell;

### Biomarker Assessments:

### Imaging Assessments:

In aspects of the disclosure, for all subjects, baseline disease assessment using PET-CT, or CT in the case of non-FDG avid disease, must be performed within 28 days prior to first dose of study drug. In aspects, imaging will be repeated on the first day of every 3rd cycle and as clinically indicated. Disease assessment may be performed by the central imaging provider. See Table 10 for examples of details and timing of disease response assessments.

### Tumor Tissue:

In aspects of the disclosure, at Screening, all subjects should provide either 14 to 15 unstained tissue sections (e.g., from a needle core, excision, or fine needle aspiration; at least 10 on charged slides, and 4 to 5 on uncharged slides, if possible) or a formalin-fixed paraffin embedded block containing sufficient tumor to cut 14 to 15 sections from their most recent biopsy, if available. The date of collection and a complete pathology report (including results of flow cytometric, cytogenetic/FISH, and molecular testing, if performed) should be submitted along with the specimen. If a subject has previously received anti-CD19 therapy, biopsy material collected after relapse or recurrence of disease after CD19 targeted therapy should be sent. In aspects, tumor samples will be analyzed at the molecular and cellular level to determine how baseline biomarker levels and changes from baseline relate to clinical outcomes, safety and resistance.

In some aspects, subjects in Part 2, Arms B and C, are asked to provide an optional fresh tumor biopsy at Screening, Cycle 3 Day 1, and progression to evaluate exploratory biomarkers (e.g., next generation sequencing to identify druggable mutations). In aspects, the aim will be to enroll 10 or more subjects consenting to these exploratory biomarker analyses, in addition to mandated biopsies/aspirates at suspected CR, and when possible at suspected progression.

In some aspects, subjects in Part 2, Arms B and C, may be asked to provide an optional fresh tumor biopsy at Screening, Cycle 3 Day 1, and progression to evaluate exploratory biomarkers (e.g., next generation sequencing to identify druggable mutations). In aspects, the aim will be to enroll 10 or more subjects consenting to these exploratory biomarker analyses, in addition to mandated biopsies/aspirates at suspected CR, and when possible at suspected progression.

TABLE 10
Disease Response Assessments

| | | | | | Off-treatment | EOT Visit^{c} |
|---|---|---|---|---|---|---|
| | | Every | | C3D1 | for reasons | If not |
| | | 3^{rd} | | and | other than | performed |
| Study | | Cycle | Suspected | Suspected | disease | within |
| Visit(s) | Screening | Day 1 | CR | PD | progression^{b} | 21 days |
| Tumor | X^{a} | | | X^{a} | | |
| Biopsy | | | | | | |
| Examination ^{d,c} | | | | | | |
| Bone | | | X | | | |
| Marrow | | | | | | |
| Examination ^{d} | | | | | | |
| PET-CT/CT^{e} | X | X | X | | X | X |

Abbreviations: CR = complete response; EOT = end of treatment; PD = progressive disease; PET-CT = positron emission tomography-computerized tomography.
^{a}Subjects in Arms B and C will be asked to provide an optional fresh tumor biopsy at Screening, C3D1 and at suspected progression.
^{b}Subjects off-treatment for reasons other than disease progression or withdrawal of consent should continue with disease response assessments every 12 weeks (±3 weeks) for the first 12 months, and then every 26 weeks (±4 weeks), until documented disease progression, initiation of subsequent anticancer therapy, or withdrawal.
^{c} The EOT visit should occur within 30 days after the last dose of TNB-486. If an alternate therapy is initiated during this period, the EOT visit should be conducted prior to the first dose of alternate therapy. Adverse events and concomitant medications/therapy should be followed for 90 days after the last dose of study drug, or until the subject begins a new line of therapy, whichever occurs sooner. Subjects will be followed for survival via telephone call every 12 weeks from the last visit. Once reliable half-life data are available for TNB-486 and if T_{1/2} exceeds 18 days, the 90 day post-last treatment visit may be performed at a time more remote from the last dose in order to capture PK and ADA data at T ≥ 5 × T_{1/2}; if this occurs, AEs will be collected until the time of this last visit or until a new line of therapy is initiated, whichever occurs earlier.
^{d} If there is suspicion of bone marrow involvement by the subject's disease at screening without a prior history, a bone marrow biopsy should be performed. If there is a suspected CR in a subject with a history of bone marrow involvement and/or when a biopsy is clinically indicated while the subject is on study, a bone marrow sample should be provided to the central laboratory for disease response assessment.
^{e}Disease assessments will be performed centrally.

### Blood Samples:

In aspects of the disclosure, blood samples will be collected from all subjects at time points indicated in the Schedule of Events (All Arms) to evaluate PK, PD and response biomarkers as well as antidrug antibodies (ADA).

In some aspects, blood samples will also be collected at designated time points throughout the study to evaluate biomarkers (i.e. cytokines and exploratory biomarkers [e.g. circulating free DNA]; Table 11). Additionally, any leftover tissue or body fluid samples taken from subjects during study or standard-of-care procedures may be utilized for the evaluation of exploratory biomarkers, with the subject's permission.

TABLE 11
Schedule of Blood Sample Collection (All Arms)

| | Cycle | | | | | | | | | | | | |
|---|---|---|---|---|---|---|---|---|---|---|---|---|---|
| | | | | Cycles 1, | | | Cycles 1 | | | | | | |
| | | All Cycles | | 3, and 6 | | | and 6 | Cycle 1 | | | | | |
| | | Day | | | | | | | | | | | |
| | | Days 1 | | Days 1 | | | | | | | | | |
| | Screening | and 15 | | and 15 | | | Day 2 | Day 3 | Day 8 | Day 16 | Day 17 | | |
| | Time ± Window | | | | | | | | | | | | |
| | | Pre- | | Post-Dose (from EOI) | | | | | | | | | |
| | | Dose | | 3 | 6 | | | | | | | | 90-day |
| | | Within | EOI | hrs ± | hrs ± | 9 | 24 | 48 | | 24 | 48 | Unsched/ | Follow- |
| | | 10 | ±10 | 10 | 10 | hrs ± | hrs ± | hrs ± | ±1 | hrs ± | hrs ± | EOT^{d} | up^{e} |
| Study Activity^{a} | N/A | min | min | min | min | 1 hr | 1 hr | 1 hr | day | 1 hr | 1 hr | N/A | N/A |
| Pharmacogenomics ^{b} | X | | | | | | | | | | | | |
| ADAs ^{c} | | X^{f} | | | | | | | | | | X | X |
| PK ^{c} | X | X | X | X | X | X | X | X | X | X | X | X | X |
| Biomarker | | X | X | X | X | X | X | X | X | X | X | X | X |
| Assessments ^{c} | | | | | | | | | | | | | |

Abbreviations: ADA = Anti-drug antibodies; EOI = end of infusion; EOT = end of treatment; min = minute(s); N/A = not applicable; PK = pharmacokinetic(s); Unsched = unscheduled.
^{f}Samples to be collected at the same time point will be included in a single blood draw if possible. Blood samples for PK, biomarker assessments and ADA testing will be shipped to a central lab.
^{g} Pharmacogenomics are optional and should only be collected at Screening. A separate consent is required.
^{h} Testing for PK, ADAs and biomarkers (e.g., cytokines, circulating free DNA) will be batch-analyzed centrally. Additional ADA testing will occur during PK sampling timepoints as appropriate.
ⁱ An unscheduled visit may occur at any time during the study. Study activities shown will be performed at the investigator's discretion.
^{j} Once adequate half-life data are available for TNB-486 and if T_{1/2} exceeds 18 days, the 90-day post-last treatment visit may be performed at a time corresponding to ~5 × T_{1/2} in order to capture PK and ADA data at T ≥ 5 × T_{1/2}.

ADAs will be collected on Day 1 of each Cycle only, except in Cycle 1 where an ADA sample will be collected on Day 15 also.

### Exploratory Research Samples:

In aspects of the disclosure, serum samples for biomarker analysis are collected from all subjects.

### Residual Blood and Tissue Samples:

In aspects of the disclosure, residual blood and tissue samples previously collected for biomarker, PK, and tissue samples may be used for optional exploratory research.

### Pharmacogenomic Samples:

In aspects of the disclosure, optional whole blood samples for DNA and RNA isolation are collected at Screening or prior to dosing on Cycle 1 Day 1.

### Activity, Pharmacokinetic, Pharmacodynamic, Immunogenicity, Pharmacogenetic and Safety Assessments/Variables:

### Activity Variables:

In aspects of the disclosure, activity will be measured by changes in target lesions according to RECIL 2017 criteria (see Table 12).

In some aspects, the activity endpoints (determined using RECIL 2017 response criteria) include objective response rate (ORR, defined as CR+PR), clinical benefit rate (CBR; defined as CR+PR+MR+SD for 24 weeks), overall survival (OS), progression-free survival (PFS), time to progression (TTP), time to response (TTR) and duration of objective response (DOR).

TABLE 12
Activity Measurements
RECIL 2017: Response Categories Based on Assessment of Target Lesions

| | % Change in Sum of Diameters of Target Lesions from Nadir | | | | |
|---|---|---|---|---|---|
| | Complete | Partial | Minor | Stable | Progression |
| | Response | Response | Response | Disease | of Disease |
| % Change | Complete | ≥30% | ≥10% | ≤10% | ≥20% |
| from | disappearance | decrease | decrease | decrease | increase |
| Baseline | of all target | in the sum | in the sum | or ≤20% | in the sum |
| | lesions and all | of longest | of longest | increase | of longest |
| | nodes with | diameters | diameters | in the sum | diameters |
| | long axis <10 mm | of target | of target | of longest | of target |
| | ≥30% | lesions but | lesions but | diameters | lesions |
| | decrease in the | not a CR | not a PR | of target | For small |
| | sum of longest | | (<30%) | lesions | lymph nodes |
| | diameters of | | | | measuring <15 |
| | target lesions | | | | mm post |
| | (PR) with | | | | therapy, a |
| | normalization | | | | minimum |
| | of FDG-PET | | | | absolute |
| | | | | | increase of |
| | | | | | 5 mm and the |
| | | | | | long diameter |
| | | | | | should exceed |
| | | | | | 15 mm |
| | | | | | Appearance |
| | | | | | of a new |
| | | | | | lesion |
| FDG-PET | Normalization | Positive | Any | Any | Any |
| | of FDG-PET | (Deauville | | | |
| | (Deauville | score 4-5) | | | |
| | score 1-3) | | | | |
| Bone Marrow | Not involved | Any | Any | Any | Any |
| Involvement | | | | | |
| New Lesions | No | No | No | No | Yes or No |

Abbreviations: CR = complete response; CT = computerized tomography; FDG-PET = [18F]2-fluoro-2-deoxy-D-glucose; PR = partial response.

### Pharmacokinetic Variables:

In aspects of the disclosure, values for the PK parameters of TNB-486, including the Cₘₐₓ, the time to Cₘₐₓ (Tₘₐₓ), the area under the concentration-time curve from time 0 to the time of the last measurable concentration (AUCₜ), CL, the terminal phase elimination rate constant, and t_{1/2} will be determined after infusion in Cycle 1 using non-compartmental methods. Additional analyses will be conducted if deemed useful and appropriate.

### Drug Concentration Measurements:

In aspects of the disclosure, the time that each blood sample is collected will be recorded to the nearest minute in the source documents and in the appropriate eCRFs. In aspects, the date and start/end time of the subject's TNB-486 infusion shall also be recorded, in the appropriate eCRFs, to the nearest minute on days when PK sampling is performed.

Pharmacokinetic timepoints may be altered slightly for the Monotherapy Dose Expansion (Part 2, Arms B and C), based on PK data from the Monotherapy Dose Escalation (Part 1, Arm A).

### Collection of Blood Samples for TNB-486 Pharmacokinetic Assay:

In aspects of the disclosure, for subject in all arms of the study, a single blood draw at the specified time points will allow for TNB-486 PK analysis (see Table 13 and below). In aspects, samples should not be drawn from the same arm in which TNB-486 is administered. In aspects, samples will be collected by venipuncture into appropriately labeled evacuated serum collection tubes at the following time points for a Q2W dosing regimen. If significant AEs are observed, additional unscheduled PK samples may be drawn.

TABLE 13

| | |
|---|---|
| Cycle 1 | Day 1: pre-dose, at the end of the infusion and 3, 6, and 9, hours after the |
| | infusion; |
| | Day 2: 24 hours after the end of the infusion on Day 1 |
| | Day 3: 48 hours after the end of the infusion on Day 1 |
| | Day 8 |
| | Day 15: pre-dose, at the end of the infusion and 3, 6, and 9, hours after the |
| | infusion; |
| | Day 16: 24 hours after the end of the infusion on Day 15 |
| | Day 17: 48 hours after the end of the infusion on Day 15 |
| Cycle 2 | Days 1 and 15: pre-dose, at the end of the infusion. |
| Cycle 3 | Days 1 and 15: pre-dose, at the end of the infusion and 3, 6, and 9, hours |
| | after the infusion; |
| Cycle 4 | Days 1 and 15: pre-dose and at the end of the infusion. |
| and | |
| Cycle 5 | |
| Cycle 6 | Day 1: pre-dose, at the end of the infusion and 3, 6, and 9, hours after the |
| | infusion; |
| | Day 2: 24 hours after the end of the infusion on Day 1 |
| | Day 15: pre-dose, at the end of the infusion and 3, 6, and 9, hours after the |
| | infusion; |
| Cycle 7 | Days 1 and 15: pre-dose and at the end of the infusion. |
| (and all | |
| subsequent | |
| cycles) | |

Unscheduled/End of Treatment (EOT) and 90 Day Follow-up

### Collection of Blood Samples for Anti-Drug Antibody (ADA) Assay:

In aspects of the disclosure, samples for ADA will be drawn before dosing TNB-486 on Day 1 of every cycle, as well as before dosing on Day 15 of cycle 1.

### Exploratory Research Variables:

In some aspects, exploratory research will be conducted to study exposure-response relationships via biomarker relationships with PK, safety, and clinical activity. In aspects, samples will be collected to conduct exploratory investigations into known and novel biomarkers. The types of biomarkers to be analyzed may include, but are not limited to, nucleic acids, proteins, lipids or metabolites. The samples may be analyzed as part of a post hoc assessment of factors influencing the subjects' response to the study drug or the development and progression of the subjects' disease or related conditions. The samples may also be used to develop new diagnostic tests, therapies, research methods or technologies.

### Safety Variables:

In aspects of the disclosure, adverse events, laboratory profiles, physical exams, and vital signs will be assessed throughout the study. In aspects, adverse events will be graded according to the NCI-CTCAE, version 5.0.

### Appropriateness of Measurements:

In aspects of the disclosure, standard PK, statistical, clinical, and laboratory procedures will be utilized in this study. In aspects, blood will also be drawn for PD markers that may add useful information towards selecting an appropriate dose of TNB-486 for future studies. Archival tissue may also be useful for selecting an appropriate dose of TNB-486 for future studies and choosing an appropriate population of subjects for treatment.

### Determination of Maximum Tolerated Dose:

In aspects of the disclosure, the MTD, if identified, will be defined at the highest dose level at which <2 of 6 subjects experience a DLT.

### Determination of Recommended Phase 2 Dose:

In aspects of the disclosure, if an MTD is reached, the RP2D will not be a dose higher than the MTD and will be selected based on the totality of the data by pooling and evaluating all available data on target-engagement, clinical PK, PD, activity, and safety of TNB-486.

### Toxicity Management:

In aspects of the disclosure, for the purpose of medical management, all AEs and laboratory abnormalities that occur during the study must be evaluated by the investigator. The table of clinical toxicity grades from the NCI-CTCAE, Version 5.0 (available on CTEP home page http://ctep.info.nih.gov) is to be used in the grading of AEs and laboratory abnormalities that are reported as AEs, each of which will be followed to satisfactory clinical resolution. TNB-486 has not been tested clinically in humans, so the AE profile in humans is not known.

In aspects, AEs should be classified as pre-existing or as Treatment-Emergent (TEAEs):
A TEAE is defined as an AE not present prior to initiation of TNB-486 treatment, or an AE present prior to TNB-486 initiation that worsens in intensity and/or frequency after initiation of TNB-486 treatment.
A pre-existing AE is an AE that does not meet these criteria. Subjects with pre-existing AEs should be assessed for any underlying illness or other causes and treated as appropriate.

In aspects, subjects with TEAE(s) should also be assessed for intercurrent illness or other causes and treated as appropriate. If a TEAE is unequivocally due to a subject's underlying malignancy or other extraneous cause, dosing with TNB-486 may be modified at the discretion of the Principal Investigator, following discussion with the Medical Monitors. If a TEAE is not unequivocally due to the subject's underlying malignancy or other extraneous cause, regardless of whether there is a ‘reasonable possibility’ that the TEAE is TNB-486-related, the dose of TNB-486 may be modified as follows:
For subjects who have completed Cycle 1 and have at least clinically or radiographically stable disease (SD, MR, PR or CR) but have experienced a reversible TEAE, the dose of study drug can be delayed for up to 28 days after the scheduled dosing date. A subject for whom dosing is delayed >28 days should be discontinued from the study.
During any cycle, if a subject develops an ANC <500/μL, platelets <10,000/μL, or a hemoglobin <6.5 g/dL, blood samples must be collected every 3 days and study treatment withheld. Treatment may resume when ANC returns to ≥1,000/μL, platelet counts return to ≥50,000/μL or baseline platelet count levels (in subjects with baseline platelets <50,000/μL), or hemoglobin returns to ≥8.0 g/dL.
TNB-486 should be discontinued in any subject who experiences CRS, TLS, or a non-hematologic TEAE that meets DLT equivalent criteria until resolution of the toxicity to ≤Grade 1 or baseline; thereafter study drug may be resumed with one dose level reduction with Medical Monitor approval.

In aspects, up to 2 dose reductions are allowed to manage toxicity; thereafter toxicity will be deemed ‘unacceptable’ and the subject will be discontinued from therapy.

While investigator discretion should be used for subject management with regards to toxicities, guidelines for management of CRS and NT are provided herein.

### Dose Limiting Toxicity (DLT) Definition for Dose Escalation:

In aspects of the disclosure, the DLT observation period for dose escalation purposes is 28 days and covers the first complete treatment cycle of TNB-486 (2 doses of TNB-486). Regular teleconferences will be conducted among the SMG to examine/confirm potential DLTs, assess AEs, and evaluate laboratory abnormalities. In aspects, events occurring outside the DLT window may be evaluated during these calls when making dose escalation decisions. In aspects, a DLT is defined as a TEAE that is not unequivocally due to the subject's underlying malignancy or other extraneous cause and meets the criteria below.

In aspects of the disclosure, DLT evaluable subjects are defined as those subjects who:
Receive at least 1 dose of TNB-486 and experience a DLT during the first 28 days
   OR
Receive at least 2 doses of TNB-486 on the Q2W schedule and are assessed for toxicities for the 28-day evaluation period

In aspects, the NCI-CTCAE version 5.0 will be used. DLT definitions are provided below.

### Non-Hematologic Dose-Limiting Toxicity:

Non-hematological AEs Grade ≥3 with the following exceptions:
   Grade 3 or 4 isolated electrolyte abnormalities (i.e. those occurring without clinical consequence) that resolve, with or without intervention, to Grade ≤1 within 72 hours.
   Grade 3 hypo/hyperglycemia responsive to optimal medical management within 72 hours.
   Grade 3 nausea/vomiting/diarrhea responsive to optimal medical management within 72 hours.
   Alopecia or vitiligo of any grade.
   Grade 3 fatigue lasting <10 days.
Any AE that requires a delay in initiation of the next scheduled cycle by >28 days.
Liver toxicity meeting Hy's criteria.

### Hematologic Dose-Limiting Toxicity:

Grade 3 CRS that does not resolve to ≤Grade 1 within 72 hours or Grade 4 CRS.
Grade 3 TLS that does not resolve to ≤Grade 1 within 72 hours or Grade 4 TLS.
Grade 4 neutropenia for >5 days or febrile neutropenia.
Grade 3 thrombocytopenia with bleeding or Grade 4 thrombocytopenia.
Grade 4 anemia.
Grade 5 AEs.
Lymphopenia will not be considered a DLT.
Adverse events that require a delay in initiation of the next scheduled cycle by >28 days.

### Cytokine Release Syndrome (CRS) Dose-Limiting Toxicity:

Cytokine release syndrome, with or without the presence of neurotoxicity, is the primary toxicity associated with T-cell redirection therapy (CARs and T-BsAbs/BiTEs). CRS occurs due to hyper-activation of the immune system and is mediated predominantly by the secretion of pro-inflammatory cytokines (most importantly IL-6 and IL-1). Signs and symptoms are those of systemic inflammation, and include the following: high fever/rigors, hypotension, hypoxia, neurologic changes, pain, nausea, and headache. Cytokine release syndrome can present with variable severity ranging from fever and flu-like symptoms, to refractory hypotension requiring high doses of vasopressors, and organ damage. Meta-analyses show that clinical findings, specifically fever, are usually the first indicators of CRS onset (Hay 2017; Wang and Han 2018).

CRS historically occurs within 14 days of first CAR/T-BsAb administration and does not usually occur in subsequent cycles. Low or selective activation by TNB-486 may delay CRS, if it occurs.

If CRS symptoms are suspected, grading should be performed to guide appropriate management. A consensus grading scheme published by Lee and colleagues is reproduced here and should be used to grade CRS (Table 14; Lee 2019). FIG. 3 provides a guideline for treatment that may be used for subject management; however, it is recommended that investigators adhere to institutional guidelines for CRS management if they exist.

TABLE 14
Guidelines for Cytokine Release Syndrome Grading, as per Lee 2019

| CRS Parameter | Grade 1 | Grade 2 | Grade 3 | Grade 4 |
|---|---|---|---|---|
| Fever^{a} | Temperature ≥38° C. | Temperature ≥38° C. | Temperature ≥38° C. | Temperature ≥38° C. |

With

| | | | | |
|---|---|---|---|---|
| Hypotension | None | Not requiring | Requiring a | Requiring |
| | | vasopressors | vasopressor with | positive pressure |
| | | | or without | (e.g., CPAP, |
| | | | vasopressin | BiPAP, |
| | | | | intubation and |
| | | | | mechanical |
| | | | | ventilation) |

And/or^{b}

| | | | | |
|---|---|---|---|---|
| Hypoxia | None | Requiring low- | Requiring | Requiring |
| | | flow nasal | high-flow | positive pressure |
| | | cannula^{c} or | nasal cannula, | (e.g., CPAP, |
| | | blow-by | facemask, | BiPAP, |
| | | | nonrebreather | intubation and |
| | | | mask, or | mechanical |
| | | | Venturi mask | ventilation) |

Abbreviations: BiPAP = bilevel positive airway pressure; CPAP = continuous positive airway pressure; CRS = cytokine release syndrome.
^{k} Fever is defined as temperature ≥38° C. not attributable to any other cause. In subjects who have CRS then receive antipyretic or anticytokine therapy such as tocilizumab or steroids, fever is no longer required to grade subsequent CRS severity. In this case, CRS grading is driven by hypotension and/or hypoxia.
^{l} CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS.
^{m} Low-flow nasal cannula is defined as oxygen delivered at 6 L/minute. Low-flow also includes blow-by oxygen delivery, sometimes used in pediatrics. High-flow nasal cannula is defined as oxygen delivered at >6 L/minute.
Source: Lee 2019

### Neurological Dose-Limiting Toxicity:

The etiology of NT is unclear but has been postulated to stem from endothelial activation/microangiopathy, possibly downstream of IL-1 secretion by monocytes/macrophages (Gust 2017; Giavidris 2018; Norelli 2018). Onset usually occurs with or after CRS (mostly CRS Grade ≥3). Isolated NT has been described after administration of anti-CD19 T-BsAbs (Velasquez 2017). Early symptoms of NT include tremor, dysgraphia, expressive aphasia, impaired attention, and lethargy; delirium, headache, agitation, cerebral edema, ataxia, confusion, seizure, and coma may subsequently develop.

If NT symptoms are suspected, grading should be performed to guide appropriate management; a consensus grading scheme published by Lee and colleagues is reproduced here and may be used to grade NT (Table 15; Lee 2019).

TABLE 15
American Society for Transplantation and Cellular Therapy Immune Effector Cell-associated
Neurotoxicity Syndrome Consensus Grading for Adults, as per Lee et al., 2019

| Neurotoxicity | | | | |
|---|---|---|---|---|
| Domain | Grade 1 | Grade 2 | Grade 3 | Grade 4 |
| ICE score^{a} | 7-9 | 3-6 | 0-2 | 0 (subject is unarousable |
| | | | | and unable to perform |
| | | | | ICE) |
| Depressed | Awakens | Awakens | Awakens only to | Subject is unarousable or |
| level of | spontaneously | to voice | tactile stimulus | requires vigorous or |
| consciousness^{b} | | | | repetitive tactile stimuli |
| | | | | to arouse. Stupor or |
| | | | | coma. |
| Seizure | NA | NA | Any clinical seizure | Life-threatening |
| | | | focal or generalized | prolonged seizure (>5 |
| | | | that resolves rapidly | min); or repetitive clinical |
| | | | or nonconvulsive | or electrical seizures |
| | | | seizures on EEG that | without return to baseline |
| | | | resolve with | in between |
| | | | intervention | |
| Motor | NA | NA | NA | Deep focal motor |
| findings^{c} | | | | weakness such as |
| | | | | hemiparesis or |
| | | | | paraparesis |
| Elevated | NA | NA | Focal/local edema | Diffuse cerebral edema |
| ICP/cerebral | | | on neuroimaging^{d} | on neuroimaging; |
| edema | | | | decerebrate or decorticate |
| | | | | posturing; or cranial |
| | | | | nerve VI palsy; or |
| | | | | papilledema; or Cushing's |
| | | | | triad |

Abbreviations: EEG = electroencephalogram; ICANS = immune effector cell-associated neurotoxicity syndrome; ICE = immune effector cell-associated encephalopathy; ICP = intracranial pressure; NA = not applicable.
Note:
ICANS grade is determined by the most severe event (ICE score, level of consciousness, seizure, motor findings, raised ICP/cerebral edema) not attributable to any other cause; for example, a subject with an ICE score of 3 who has a generalized seizure is classified as Grade 3 ICANS.
^{a}A subject with an ICE score of 0 may be classified as Grade 3 ICANS if awake with global aphasia, but a subject with an ICE score of 0 may be classified as Grade 4 ICANS if unarousable.
^{b}Depressed level of consciousness should be attributable to no other cause (e.g., no sedating medication).
^{c}Tremors and myoclonus associated with immune effector cell therapies may be graded according to CTCAE v5.0, but they do not influence ICANS grading.
^{d}Intracranial hemorrhage with or without associated edema is not considered a neurotoxicity feature and is excluded from ICANS grading. It may be graded according to CTCAE v5.0.
Source: Lee et al., 2019

Neurological AEs in subjects receiving TNB-486 mandate frequent CAR-T-cell therapy associated toxicity (CARTOX-10) or immune effector cell encephalopathy (ICE) examinations (Table 16) and neurological exams. Early neurology consultation, use of anti-epileptics, and intensive care unit/airway support as needed are encouraged. Table 17 provides a guideline for treatment that may be used for subject management, however, it is recommended that investigators adhere to institutional guidelines for CRS management, if they exist.

TABLE 16
Encephalopathy Assessment Tools for Grading of Immune Effector
Cell-associated Neurotoxicity Syndrome, as per Lee 2019

| CARTOX-10 | ICE |
|---|---|
| Orientation: orientation to year, | Orientation: orientation to year, month, |
| month, city, hospital, president/prime | city, hospital: 4 points |
| minister of country of residence: 5 | Naming: ability to name 3 objects (e.g., |
| points | point to clock, pen, button): 3 points |
| Naming: ability to name 3 objects | Following commands: ability to follow |
| (e.g., point to clock, pen, button): 3 | simply commands (e.g., “Show me |
| points | 2 fingers” or “Close your eyes and stick |
| | out your tongue”): 1 point |
| Writing: ability to write a standard | Writing: ability to write a standard |
| sentence (e.g., Our national bird is the | sentence (e.g., Our national bird is the |
| bald eagle”): 1 point | bald eagle”): 1 point |
| Attention: ability to count backwards | Attention: ability to count backwards |
| from 100 by 10: 1 point | from 100 by 10: 1 point |

Abbreviations: CARTOX-10 = chimeric antigen receptor toxicity; ICE = immune effector cell encephalopathy.
Note:
CARTOX-10 (left column) has been updated to the ICE tool (right column). ICE adds a command-following assessment in place of 1 of the CARTOX-10 orientation questions. The scoring system remains the same.
Note:
Scoring: 10, no impairment; 7-9, Grade 1 ICANS; 3-6, Grade 2 ICANS; 0-2, Grade 3 ICANS; 0 due to subject unarousable and unable to perform ICE assessment, Grade 4 ICANS.
Source: Lee 2019

TABLE 17
Suggested Guidelines for Management of Neurological Toxicity

| AE Category | Guidelines |
|---|---|
| Neurologic AE | Consult neurologist |
| after dosing with | Continue neurologic monitoring |
| TNB-486 | Prophylactic anti-epileptic (i.e., levetiracetam) |
| | Consider MRI/CT, EEG, and/or LP |
| | Contact Medical Monitor |
| If AE is Grade ≥2, | Consider Corticosteroids (e.g., dexamethasone 10 to 20 mg IV |
| add | every 12 to 24 hours, higher doses or with more frequency, if |
| | clinically indicated) |
| | Withhold TNB-486 until resolution to Grade ≤1. |
| If AE is Grade ≥3, | High-dose corticosteroids (methylprednisolone 2 mg/kg |
| add | loading dose followed by 2 mg/kg/day divided 4 times per |
| | day). Higher/more frequent doses if indicated. Taper when |
| | Grade 1 or better) |
| | Anti-epileptics for seizures/seizure-like activity |
| | ICU monitoring |
| | Airway protection |
| | Consider continuous EEG monitoring |
| If AE is Grade 4, | If cerebral edema/other Grade 4 toxicity is rapid onset or the |
| add | subjects is unresponsive to above: methylprednisolone IV |
| | 1 g/day × 3 days, followed if clinically indicated by taper. |
| | Consider hyperventilation/hyperosmolar therapy for cerebral |
| | edema. |

Abbreviations: AE = adverse event; CT = computed tomography; EEG = electroencephalogram; ICU = intensive care unit; IV = intravenous; LP = lumbar puncture; MRI = magnetic resonance imaging.

### Miscellaneous Dose-Limiting Toxicity:

Any toxicity thought to be related to the study drug that, at the discretion of the investigator, is thought to warrant withholding the drug. Other ALs may be considered a DLT as determined by the TeneoTwo Medical Monitor, in conjunction with the investigator.

All decisions regarding continued dosing for individual subjects will be medically managed by the investigator, in conjunction with the Medical Monitor(s), as appropriate. These decisions will be driven by the DLT criteria as described above.

Any subject who does not complete the full 28-day DLT observation period for any reason other than DLT will be considered non-DLT-evaluable for dose escalation and/or MTD assessment and will be replaced at the same dose level. Additional subjects may be enrolled at a given dose level in the absence of DLT to explore factors influencing AEs or to accumulate additional safety data.

### Activity Analysis:

In aspects of the disclosure, for the cohorts in the Monotherapy Dose Escalation (Part 1, Arm A), activity data will be listed. For the Monotherapy Dose Expansion (Part 2, Arms B and C) activity analyses will be performed based on the EE population and Safety Population as appropriate. In aspects, subject response and disease progression will be determined using the RECIL 2017 criteria.

In aspects of the disclosure, objective response rate, DOR, PFS and CBR will be summarized and listed for both the Monotherapy Dose Escalation (Part 1, Arm A) and the Monotherapy Dose Expansion (Part 2, Arms B and C). Kaplan-Meier estimates for PFS and associated CI of the median PFS, OS, and TTP will be provided. In addition, in some aspects, descriptive summaries for the ORR and its DOR, and CBR along with the associated CIs will be provided. In aspects, for the Monotherapy Dose Expansion (Part 2, Arms B and C), activity analyses will be performed based on the Efficacy Evaluable Population and repeated for the Safety Population unless the sample size of the 2 populations is the same. In addition, in some aspects, results from the Monotherapy Dose Escalation (Part 1, Arm A) for the MTD or RP2D may be pooled with results from the Monotherapy Dose Expansion (Part 2, Arms B and C) for the analyses as appropriate.

### Objective Response Rate (ORR):

In aspects of the disclosure, objective response rate is defined as the proportion of subjects with a confirmed partial or complete response to treatment, and is sometimes referred to as the overall response rate. In aspects, the ORR for each dose cohort will be estimated with all the sites pooled. In aspects, the 2-sided 80% and 90% exact binomial CIs of ORR will also be summarized using the Clopper-Pearson method along with the best overall response (CR, PR, SD, PD).

### Progression-Free Survival (PFS):

In aspects of the disclosure, progression-free survival time is defined as the time from the first dose of TNB-486 to progression or death, whichever occurs first. In aspects, subjects will be censored at the date of last tumor assessment if neither event occurred. In aspects, the Kaplan-Meier method will be used to analyze PFS.

### Duration of Objective Response (DOR):

In aspects of the disclosure, the duration of objective response for a subject is defined as the time from the initial objective response to disease progression or death, whichever occurs first. In aspects, if the subject did not progress or die then the subject will be censored at the date of the last tumor assessment similar to the censoring rules for the PFS analysis.

In aspects, the DOR will be analyzed in the same fashion as for the PFS analysis.

### Clinical Benefit Rate (CBR):

In aspects of the disclosure, the clinical benefit rate is defined as the proportion of subjects with a confirmed CR, PR or MR, or SD for at least 24 weeks after responding to treatment. In aspects, the CBR for each arm will be estimated with all the sites pooled. In aspects, the 2-sided 80% exact binomial CIs of the CBR will also be summarized using the Clopper-Pearson method.

### Clinical Laboratory Tests:

In aspects of the disclosure, baseline laboratory tests will be performed at the central laboratory for the study. In aspects, all disease response assessment laboratory tests from subsequent time points will be performed at the central lab. In aspects, all samples for PK, ADA, and biomarker assessment will also be managed by the central lab. In aspects, all other lab tests will be performed in the local labs associated with the clinical sites.

In aspects of the disclosure, changes from baseline in clinical laboratory results will be summarized by dose cohort and time point using descriptive statistics. In aspects, summaries of shifts from baseline to last available visit will be provided. In aspects, shifts will be calculated as the proportion of subjects at baseline with values that are below, within, or above the normal range for a particular lab test relative to the proportion of subjects at the Final Visit with values that are below, within, or above the normal range.

In aspects of the disclosure, lab abnormalities and treatment-emergent lab abnormalities meeting the NCI-CTCAE version 5.0 will be summarized by treatment arm and overall.

### Eastern Cooperative Oncology Group (ECOG) Performance Scores:

In aspects of the disclosure, the ECOG performance scores will be listed in a subject data listing and summarized by visit and change from baseline by visit.

### Vital Signs and Physical Exams:

In aspects of the disclosure, changes from baseline in vital signs and physical examination findings will be summarized by dose cohort and time point using descriptive statistics.

### Pharmacokinetics:

### Tabulations and Summary Statistics:

In aspects of the disclosure, serum concentrations of TNB-486 and PK parameter values will be tabulated for each subject, each dose level, and summary statistics will be computed for each sampling time and each parameter.

### Dose Proportionality Analysis:

In aspects of the disclosure, pharmacokinetic parameters of TNB-486 from a particular dosing schedule assessed on Cycle 1 Day 1 will be analyzed as follows. An analysis will be performed for dose normalized Cmax and dose-normalized AUC provided that they can be adequately determined from the data. The model used for the statistical analyses will include the dose level of TNB-486 as a categorical variable. Covariates such as age, ethnicity, gender, and perhaps others that might explain some of the variability in the population may be included in the initial model. However, a covariate may be dropped from the model if the regression coefficient is not significant at alpha level 0.10.

In aspects, the natural logarithmic transformation will be employed for Cmax and the AUCs unless the data clearly indicate that another transformation or the untransformed variable provides more nearly symmetric probability distributions and/or more nearly homogenous variances across dose levels. In aspects, assuming that at least 3 dose levels of TNB 486 are studied, a test will be performed on a contrast in the dose level effects chosen to be sensitive to an approximately linear function of dose or the logarithm of dose.

Additional analyses will be performed if useful and appropriate.

### Missing Values and Model Violations:

In aspects of the disclosure, all available data will be included in the dose proportionality analysis. Data exclusion, if any, will be documented and justification provided.

In aspects, normally values of PK variables (Cₘₐₓ, AUC, etc.) are determined without replacing missing individual concentration values, by simply using the available data. However, if a missing individual concentration results in a PK parameter value that may be too low or too high to a meaningful degree, the value of the PK parameter will tentatively be considered missing. In this case, a value for the missing individual concentration may be imputed so that an appropriate value of the PK parameter can be included in the analysis. In aspects, the imputed value will be obtained using appropriate methodology that considers the individual characteristics of the subject.

In aspects, if an outlier is identified and/or a pronounced non-normal probability distribution is observed (after logarithmic transformation for Cₘₐₓ and AUC) then a non-parametric analysis may also be performed. Such a model violation may be identified by graphical methods, measures of non-normality (e.g., skewness, kurtosis) or other appropriate methods. If different dose levels have unequal variances to the extent that conclusions might be affected, then approximate methods that allow for unequal variances will be used. The possibility of bias from missing data of subjects who prematurely discontinue due to an adverse event will be addressed.

### Pharmacodynamics/Biomarkers:

In aspects of the disclosure, the biomarker analyses are exploratory. In aspects, descriptive statistics of the baseline, post-baseline, and change from baseline biomarkers will be summarized by measurement time point/visit and listed. In addition, exploratory analyses will be performed to evaluate the association of each biomarker or combination of biomarkers with clinical outcomes, the modulation of biomarkers related to mechanism of action, and biomarker or combination of biomarkers potentially predictive of treatment response.

### V. Pharmaceutical Compositions for Use

Aspects of the disclosure include pharmaceutical compositions for treating B-cell non-Hodgkin lymphoma in a patient, the pharmaceutical compositions comprising a therapeutically effective amount of TNB-486, wherein the TNB-486 is administered according to a 28-day treatment cycle, wherein the therapeutically effective amount of TNB-486 in the pharmaceutical composition is from about 30 μg to about 30000 μg.

In some aspects of the pharmaceutical composition for use, the treatment cycle is repeated two or more times. In some aspects, TNB-486 is administered to the patient as a monotherapy. In some aspects, TNB-486 is administered by an intravenous infusion (IV). In some aspects, the patient has received at least two prior lines of systemic therapy. In some aspects, the patient is CD19-positive. In some aspects, the patient has an Eastern Cooperative Oncology Group (ECOG) performance status of less than or equal to 2. In some aspects, the patient has adequate bone marrow function. In some aspects, the patient has an estimated glomerular filtration rate (eGFR) greater than or equal to 50 mL/min. In some aspects, the patient has a total bilirubin of less than or equal to 1.5 times an upper limit of normal, an aspartate aminotransferase (AST) of less than or equal to 3 times an upper limit of normal, and an alanine amino transferase (ALT) of less than or equal to 3 times an upper limit of normal.

Aspects of the disclosure include pharmaceutical compositions for improving an objective response rate (ORR) in a patient diagnosed with B-cell non-Hodgkin lymphoma, the pharmaceutical compositions comprising a therapeutically effective amount of TNB-486 administered according to a 28-day treatment cycle, wherein the therapeutically effective amount of TNB-486 is from about 30 μg to about 30000 μg.

Aspects of the disclosure include pharmaceutical compositions for improving an overall survival (OS) rate in a patient diagnosed with B-cell non-Hodgkin lymphoma, the pharmaceutical compositions comprising a therapeutically effective amount of TNB-486 administered according to a 28-day treatment cycle, wherein the therapeutically effective amount of TNB-486 is from about 30 μg to about 30000 μg.

Aspects of the disclosure include pharmaceutical compositions for improving a progression free survival (PFS) rate in a patient diagnosed with B-cell non-Hodgkin lymphoma, the pharmaceutical compositions comprising a therapeutically effective amount of TNB-486 administered according to a 28-day treatment cycle, wherein the therapeutically effective amount of TNB-486 is from about 30 μg to about 30000 μg.

Aspects of the disclosure include pharmaceutical compositions for improving a time to progression (TTP) in a patient diagnosed with B-cell non-Hodgkin lymphoma, the pharmaceutical compositions comprising a therapeutically effective amount of TNB-486 administered according to a 28-day treatment cycle, wherein the therapeutically effective amount of TNB-486 is from about 30 μg to about 30000 μg.

Aspects of the disclosure include pharmaceutical compositions for improving a time to response (TTR) in a patient diagnosed with B-cell non-Hodgkin lymphoma, the pharmaceutical compositions comprising a therapeutically effective amount of TNB-486 administered according to a 28-day treatment cycle, wherein the therapeutically effective amount of TNB-486 is from about 30 μg to about 30000 μg.

Aspects of the disclosure include pharmaceutical compositions for improving a duration of objective response (DOR) in a patient diagnosed with B-cell non-Hodgkin lymphoma, the pharmaceutical compositions comprising a therapeutically effective amount of TNB-486 administered according to a 28-day cycle, wherein the therapeutically effective amount of TNB-486 is from about 30 μg to about 30000 μg.

Aspects of the disclosure include pharmaceutical compositions for improving a clinical benefit rate (CBR) in a patient diagnosed with B-cell non-Hodgkin lymphoma, the pharmaceutical compositions comprising a therapeutically effective amount of TNB-486 administered according to a 28-day cycle, wherein the therapeutically effective amount of TNB-486 is from about 30 μg to about 30000 μg.

In some aspects of the pharmaceutical composition for use, the improvement is at least about 5%, at least about 10%, at least about 15%, at least about 20%, at least about 25%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or at least about 100%.

In some aspects of the pharmaceutical composition for use, the treatment cycle is modified to add more time between doses. In some aspects, the treatment cycle is modified by consistently eliminating one or more treatment cycles from a dosing regimen.

In some aspects of the pharmaceutical composition for use, the treatment cycle is modified to include a priming dose. In some aspects, the priming dose is from about 150 μg to about 1500 μg. In some aspects, the priming dose is from about 270 μg to about 1000 μg. In some aspects, the priming dose is administered at a first timepoint in the first treatment cycle, and a full dose is administered at all subsequent timepoints. In some aspects, the priming dose is administered on day 1 of the first treatment cycle, the full dose is administered on day 15 of the first treatment cycle, and the full dose is administered on days 1 and 15 of all subsequent treatment cycles.

In some aspects of the pharmaceutical composition for use, the treatment cycle is modified to include at least two priming doses. In some aspects, the first priming dose is from about 150 μg to about 540 μg. In some aspects, the second priming dose is from about 800 μg to about 1200 μg. In some aspects, the first priming dose is about 270 μg and the second priming dose is about 1000 μg. In some aspects, the first priming dose is administered on day 1 of the first treatment cycle, the second priming dose is administered on day 8 of the first treatment cycle, the full dose is administered on day 15 of the first treatment cycle, and the full dose is administered on days 1 and 15 of all subsequent treatment cycles.

In some aspects of the pharmaceutical composition for use, the full dose is equal to or less than 100% more than a full dose corresponding to a next lowest dose cohort. In some aspects, the full dose is equal to or less than 50% more than a full dose corresponding to a next lowest dose cohort. In some aspects, the full dose is equal to or less than 33% more than a full dose corresponding to a next lowest dose cohort.

In some aspects of the pharmaceutical composition for use, the patient is premedicated prior to administration of TNB-486 with an agent that reduces a risk or severity of a hypersensitivity reaction. In some aspects, the agent that reduces the risk or severity of a hypersensitivity reaction is selected from the group consisting of: dexamethasone, diphenhydramine, acetaminophen, ranitidine, tocilizumab, any equivalents thereof, or any combination thereof. In some aspects, the agent that reduces the risk or severity of a hypersensitivity reaction is administered 15-60 minutes prior to administration of TNB-486. In some aspects of the pharmaceutical composition for use, the therapeutically effective amount of TNB-486 is about 30 μg, 90 μg, 270 μg, 800 μg, 2400 μg, 7200 μg, 15000 μg, or 30000 μg.

### Example 1

Results of the Phase 1 Study of TNB-486 in Patients with Relapsed Refractory B-NHL

### Background:

TNB-486 is a novel CD19xCD3 bispecific T-cell engager (TCE) that incorporates a unique anti-CD3 moiety designed to reduce cytokine release syndrome by binding to T-cells with low affinity. A silenced lgG4 backbone confers a long half-life suitable for intermittent administration. A combination of TNB-486, which targets CD19, with R-CHOP, which incorporates CD20 targeting via Rituxan, in DLBCL may lead to synergistic tumor kill and reduce the risk of antigen escape, offering a promising approach for improved long-term remission. Here, interim results of the ongoing first in human (FIH) phase 1 study of TNB-486 in R/R 8-NHL are presented.

### Methods:

The primary objectives of the study were to assess the safety, tolerability and pharmacokinetics of TNB-486 when administered as monotherapy and to determine the optimal biologically active dose. Patients with R/R 8-NHL after at least 2 prior lines of therapy were eligible; prior anti-CD19 therapy was permitted. Patients received escalating doses of TNB-486 infused IV over 1-2 hours Q2W until PD/unacceptable toxicity. Fixed doses were given initially at lower doses, followed by a priming dose at higher target doses (>2.4 mg). Responses were assessed by RECIL 2017 and adverse events were graded using CTCAE, except for cytokine release syndrome (CRS) and neurotoxicity (NT) which were graded according to ASTCT criteria.

### Results:

As part of the current study for a first clinical cutoff, 27 subjects have received TNB-486 at doses from 0.030-10 mg. 22 patients were evaluable for efficacy and 27 for safety. Patient characteristics are summarized in Table 18. Overall, patients were heavily pre-treated, with a median of 4 prior lines of therapy (ranging from 2-21 prior lines) and 19% of enrolled subjects had previously failed CAR-T.

Enrollment was expanded and 30 patients were evaluated for a second clinical cutoff, with 25 evaluable for efficacy and 30 for safety. Patient characteristics are summarized in Table 19. For the second clinical cutoff, 23% of enrolled subjects had previously failed CAR-T.

TABLE 18
Summary of Enrolled Patients for First Clinical Cutoff
Gender

| | | |
|---|---|---|
| Female | 12 | (44) |
| Male | 15 | (56) |
| Age, Median (Range) | 68 | (38-85) |

Lymphoma Subtype, n (%)

| | | |
|---|---|---|
| Follicular | 12 | (44) |
| Diffused Large B-cell/High Grade | 7 | (26) |
| Marginal Zone | 4 | (15) |
| Mantle Cell | 4 | (15) |
| Prior Lines of Therapy, Median (Range) | 4 | (2-21) |
| >3 Prior Lines of Therapy | 15 | (56) |
| Prior CAR-T | 5 | (19) |
| Prior HSCT | 4 | (15) |

Lymphoma Stage at Diagnosis, n (%)

| | | |
|---|---|---|
| Unknown | 3 | (11) |
| Stage I-II | 3 | (11) |
| Stage III-IV | 21 | (78) |

ECOG, n (%)

| | | |
|---|---|---|
| 0 | 8 | (30) |
| 1 | 17 | (63) |
| 2 | 2 | (7) |
| CD20-negative, n (%) | 6 | (22) |

TABLE 19
Summary of Enrolled Patients for Second Clinical Cutoff

| | N = 30 | |
|---|---|---|
| Median age, years (range) | 68 | (33-88) |
| ECOG PS 0/1/2 | 9/18/3 | |
| DLBCL | 7 | (23%) |
| FL | 13 | (43%) |
| MCL | 5 | (17%) |
| MZL | 4 | (13%) |
| Richter's transformation | 1 | (3%) |
| CD20 negative disease at study entry | 7/30 | (23%) |
| Ann Arbor stage, n (%) | | |
| I-II | 4 | (13%) |
| III-IV | 23 | (77%) |
| Missing | 3 | (10%) |
| Median lines of prior therapy, n (range) | 4 | (2-21) |
| Prior CART-19 | 7 | (23%) |
| Prior auto-HSCT | 3 | (10%) |
| Prior allo-HSCT | 1 | (3%) |

For the first clinical cutoff, only one case of Gr 3 CRS was observed, and no CRS (any grade) occurred post-cycle 1. Median time to onset of CRS was 1 day with a median time to resolution of 1.5 days (ranging from <1-9). Eight subjects received tocilizumab; six of these had Gr2 CRS. Six subjects (22%) experienced NT (Grade 3 700 no Grade 4, occurring in two subjects with MZL and Richter's transformation, respectively), all were transient and resolved without sequelae. No Gr2 or higher NT was reported in DLBCL or FL, and no new case of NT occurred beyond cycle 1. Four subjects received steroids for NT. Notably, 1 subject experiencing Gr3 NT continued therapy at a reduced dose with no recurrence.

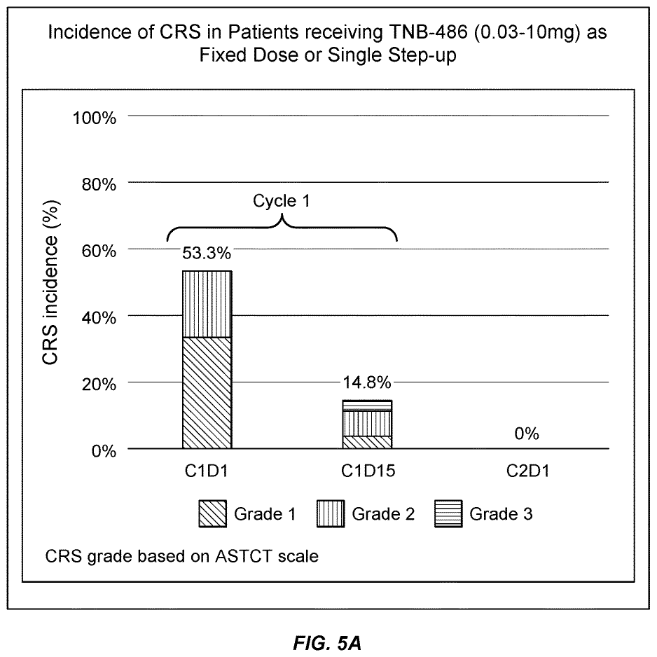

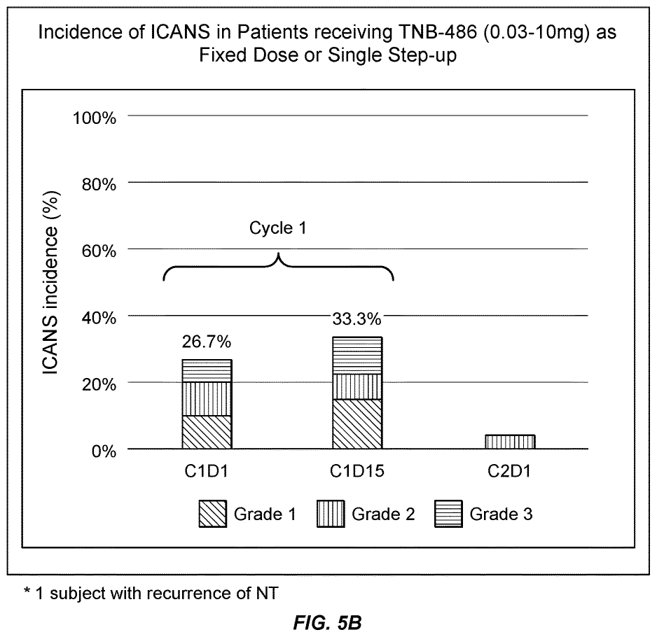

CRS results were very similar at the second clinical cutoff, as shown in Table 20 and FIG. 5A.

TABLE 20
Summary of CRS Events for Second Clinical Cutoff

| | N = 30 | |
|---|---|---|
| Patients that experienced CRS | 18 | (60%) |
| Grade 1 | 11 | (37%) |
| Grade 2 | 6 | (20%) |
| Grade 3 | 1 | (3%) |
| Onset, median (range) | 1 | (0-2) |
| Duration, median (range) | 2 | (0-9) |
| Tocilizumab use for CRS | 9 | (30%) |
| Resolved | 18 | (100%) |

Immune Effector Cell-Associated Neurotoxicity Syndrome (ICANS) was evaluated at the second clinical cutoff, with the results shown in Table 21 and FIG. 5B. Events classified as neurotoxicity were reported by 10/30 subjects (330%). All of these events resolved. Grade 3 events occurred in 4 patients with risk factors (e.g. age >80 yrs, aggressive histology and/or prior G4 event post-CART). Three out of these 4 patients was subsequently re-challenged without recurrence.

TABLE 21
Summary of ICANS Events for Second Clinical Cutoff

| | N = 30 | |
|---|---|---|
| Patients that experienced symptoms consistent with ICANS | 10 | (33%) |
| Grade 1 | 4 | (13%) |
| Grade 2 | 2 | (6%) |
| Grade 3 | 4 | (13%) |
| Resolved | 10 | (100%) |

Preliminary PK data showed an average T_{1/2} that ranged from 7.55-11.7 days at active doses, supporting the Q2W (or less frequent) dosing of TNB-486.

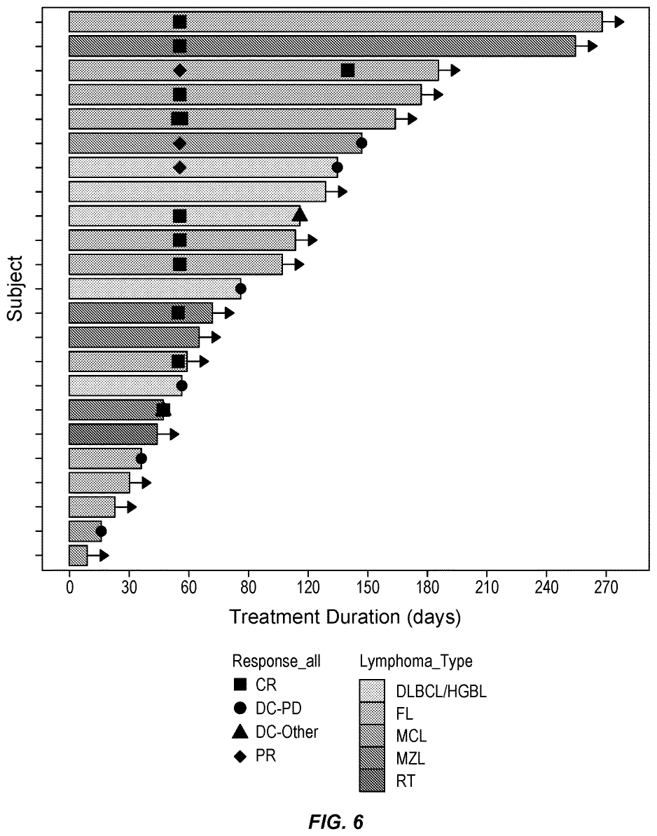

First Clinical Cutoff: At a first clinical cutoff date, the overall response rate (ORR) at 800 μg was 72% (13/18 evaluable subjects), with a CR rate of 61% (11/18). Among response-evaluable CAR-T exposed subjects, 2/3 achieved a CR. Among response-evaluable FL subjects treated at 800 μg, the ORR was 88% (7/8 subjects); all responding subjects achieved a CR. Five DLBCL subjects were treated at 800 μg with an ORR of 40%; one subject achieved an MRD-negative CR. Notably, this subject had been previously treated with 5 lines of therapy including CAR-T and never achieved a CR prior to this study. Among 4 subjects with MZL, the ORR was 75% (all CR). At a median follow-up of 3.8 months (range: 1.5-8.7 months), no subjects in CR have relapsed, with ongoing remission at up to 10 months post-initiation of TNB-486 therapy (FIG. 6). Responses were seen across all NHL subtypes and prognostic factors (e.g. disease burden, prior lines of therapy, and refractoriness to prior therapy).

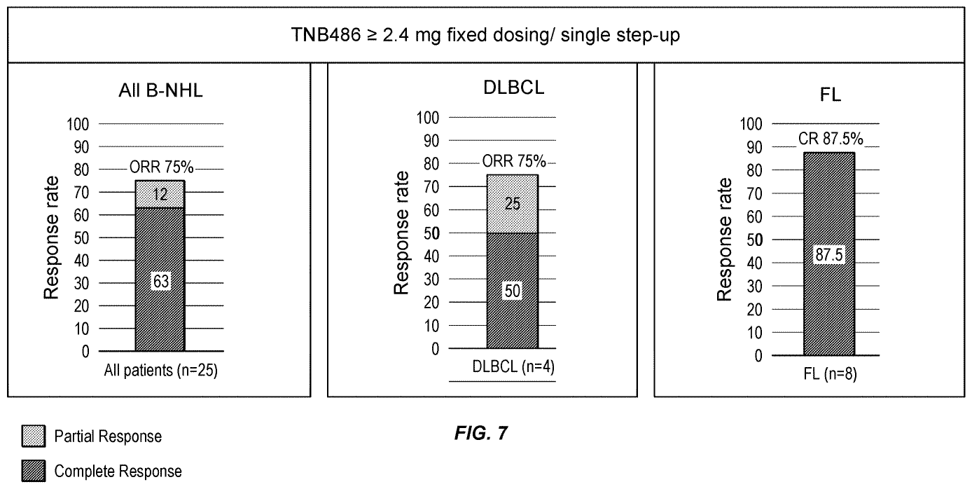

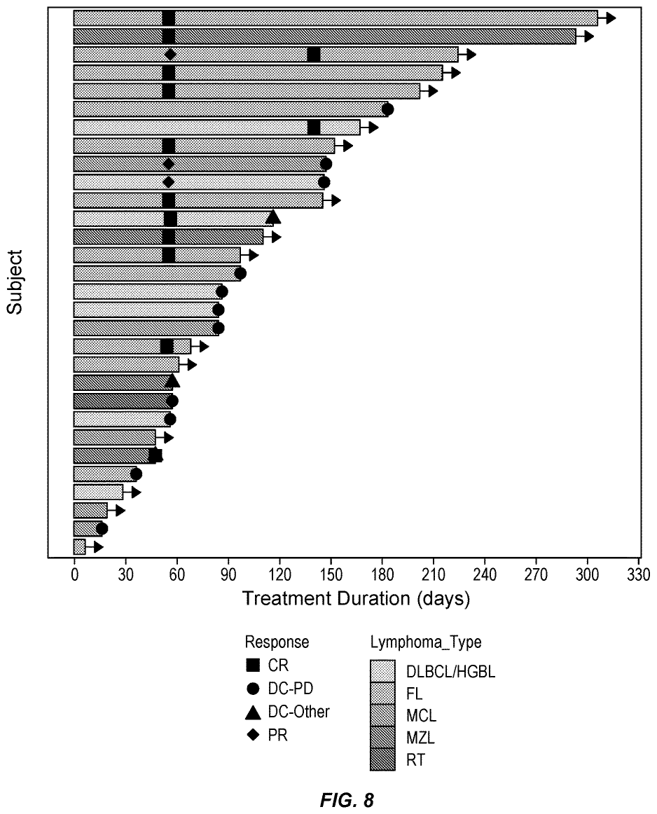

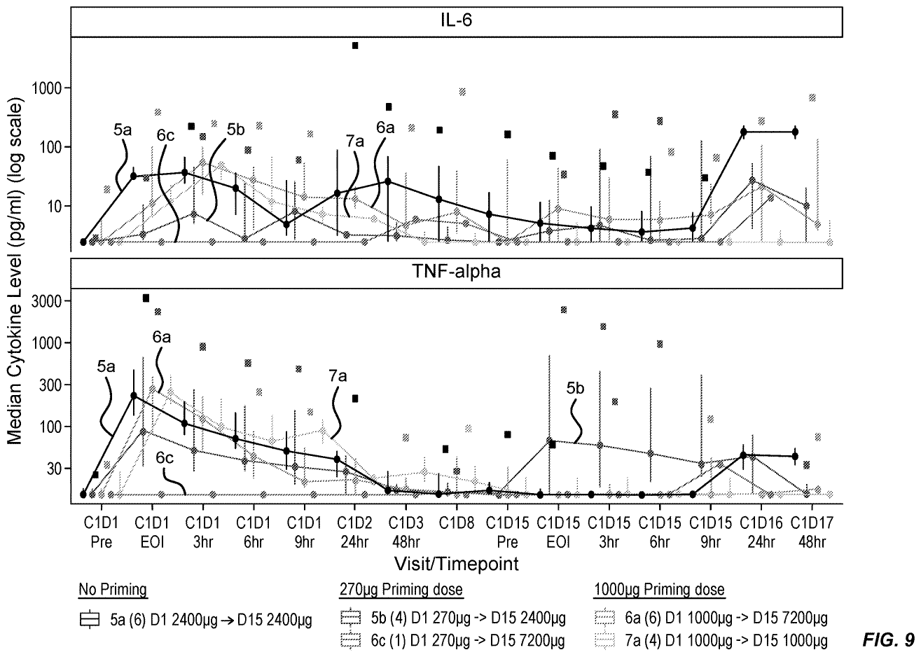

Second Clinical Cutoff: At the second clinical cutoff date, the overall response rate (ORR) was greater than 75% (19/25 evaluable subjects), with a CR rate of 63% (16/25). Among response-evaluable CAR-T exposed subjects, 2/4 achieved a CR. Among response-evaluable FL subjects, the ORR was 88% (7/8 subjects) and all responding subjects achieved a CR. See FIG. 7. At a median follow-up of 3.8 months (range: 1.5-8.7 months), no subjects in CR have relapsed, with ongoing remission at up to 10 months post-initiation of TNB-486 therapy (FIG. 8). Responses were seen across all NHL subtypes and prognostic factors (e.g. disease burden, prior lines of therapy, and refractoriness to prior therapy). Preliminary data from the current FIH study of TNB-486 show a tolerable safety profile with mostly low-grade CRS/NT at doses up to 7.2 mg (dose escalation ongoing) and promising activity in heavily pretreated B-NHL.

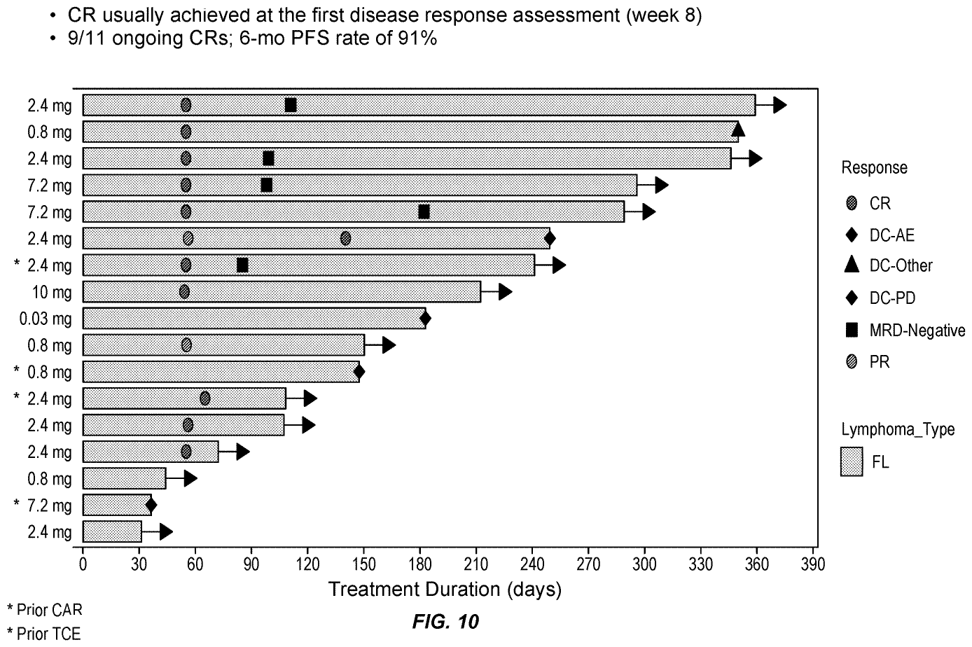

Third Clinical Cutoff: At the third clinical cutoff date, 17 pts received TNB-486 at target doses 0.03-10 mg (median age 68 y [range 33-86]; 53% male; 65% stage III/IV; 25% CD20-disease; median prior lines of therapy [LOT]3 [range 2-9]). Prior therapies included αCD20 Ab (100%), alkylator (76%), IMiD (47%), CD20 TCE (12%), CD19 CAR T (12%), and ASCT (6%); 53% progressed or started 2nd LOT within 24 months of initiating 1st LOT (POD24). Median time on study was 7 mo (range 1-22). Eleven pts were evaluable for efficacy at target doses ≥2.4 mg. Objective response rate (ORR) and complete response (CR) rate was 91% as seen in FIG. 10. ORR/CR for pts with CD20− disease, prior CD20 TCE, and POD24 was 100%. For pts with CR, 1 with 6 prior LOT progressed at C6 with preserved CD19 expression. The 6-mo PFS rate was 91%. No G3+ CRS occurred (59% G1, 12% G2). Neurologic events consistent with ICANS were reported in 24%, with 1 G3 event (confusion). All CRS/NT were transient, resolving in a median of 1.5 d (range 1-5). G3+ treatment-related AEs in >10% of pts included decreased lymphocytes (35%) and neutropenia (12%).

### Example 2

### Follow-on Results of Phase I Study

This example provides additional data obtained during Part 1, Arm A of the Phase I study described in Example 1 above. A summary of the Cohorts in Arm A is provided in Table 22.

TABLE 22
Summary of Arm A Cohorts

| | | | |
|---|---|---|---|
| | Number of | Priming Dose | Dose |
| Cohort | Subjects | (ug) | (ug) |

Fixed Dose

| | | | |
|---|---|---|---|
| 1 | 1 | NA | 30 |
| 2 | 2 | NA | 90 |
| 3 | 1 | NA | 270 |
| 4a | 3 | NA | 800 |
| 5a | 6 | NA | 2400 |

Single Priming Dose

| | | | |
|---|---|---|---|
| 4b | 4 | 270 | 800 |
| 5b | 13 | 270 | 2400 |
| 6c | 1 | 270 | 7200 |
| 6a | 8 | 1000 | 7200 |
| 7a | 4 | 1000 | 10000 |

Double Step-Up Priming Dose

| | | | |
|---|---|---|---|
| 5d | 8 | 270 + 1000 | 2400 |
| 6d | 13 | 270 + 1000 | 7200 |

For each Cohort, overall response, objective response rate and clinical benefit rate were evaluated as described above. A summary of responses for each Cohort provided in Table 23. Abbreviations are as defined above.

TABLE 23
Summary of Responses In Each Cohort

| | Best Overall Response[1] n (%) | | | | | | | |
|---|---|---|---|---|---|---|---|---|
| | | | | | | | ORR[2] n | CBR [3] n |
| Cohort | CR | PR | MR | SD | PD | NE | (%) | (%) |
| 1 | 0 | 0 | 0 | 1 | 0 | 0 | 0 | |
| | | | | (100.0) | | | | |
| 2 | 0 | 0 | 0 | 0 | 2 | 0 | 0 | |
| | | | | | (100.) | | | |
| 3 | 0 | 0 | 0 | 1 | 0 | 0 | 0 | |
| | | | | (100.0) | | | | |
| 4a | 2 | 0 | 0 | 0 | 1 | 0 | 2 | 2 |
| | (66.7) | | | | (33.3) | | (66.7) | (66.7) |
| 5a | 4 | 1 | 0 | 0 | 1 | 0 | 5 | 5 |
| | (66.7) | (16.7) | | | (16.7) | | (83.3) | (83.3) |
| 4b | 2 | 1 | 1 | 0 | 0 | 0 | 3 | 4 |
| | (50.0) | (25.0) | (25.0) | | | | (75.0) | (100.0) |
| 5b | 4 | 1 | 0 | 0 | 4 | 0 | 5 | 5 |
| | (30.8) | (7.7) | | | (30.8) | | (38.5) | (38.5) |
| 6c | 0 | 0 | 0 | 0 | 0 | 0 | 0 | 0 |
| 6a | 5 | 0 | 0 | 0 | 3 | 0 | 5 | 5 |
| | (62.5) | | | | (37.5) | | (62.5) | (62.5) |
| 7a | 2 | 0 | 0 | 0 | 2 | 0 | 2 | 2 |
| | (50.0) | | | | (50.0) | | (50.0) | (50.0) |
| 5d | 5 | 2 | 1 | 0 | 0 | 0 | 7 | 8 |
| | (62.5) | (25.0) | (12.5) | | | | (87.5) | (100.0) |
| 6d | 4 | 1 | 0 | 2 | 5 | 0 | 5 | 5 |
| | (30.8) | (7.7) | | (15.4) | (38.5) | | (38.5) | (38.5) |

[1]Best overall response is defined as the best response observed post-treatment and prior to the start of any new anti-cancer therapy for each subject.
[2]ORR is defined as proportion of subjects with a confirmed partial or complete response to treatment as determined by RECIL 2017.
[3] CBR is defined as the proportion of subjects with a confirmed CR, PR or MR, or SD for at least 24 weeks after responding to treatment as determined by RECIL 2017.

A summary of responses for All Fixed and Single Priming Dose Cohorts, All Double Step-up Priming Dose Cohorts and All Subjects combined is provided in Table 24. Abbreviations are as defined above, the same as for Table 23.

TABLE 24
Combined Summary of Responses

| | | |
|---|---|---|
| All Fixed and Single | All Double Step-up | All |
| Priming Cohorts | Priming Cohorts | Subjects |
| (n = 43) | (n = 21) | (n = 64) |

Best Overall Response, n (%)

| CR | 19 | (44.2) | 9 | (42.9) | 28 | (43.8) |
|---|---|---|---|---|---|---|
| PR | 3 | (7.0) | 3 | (14.3) | 6 | (9.4) |
| MR | 1 | (2.3) | 1 | (4.8) | 2 | (3.1) |
| SD | 2 | (4.7) | 2 | (9.5) | 4 | (6.3) |
| PD | 13 | (30.2) | 5 | (23.8) | 18 | (28.1) |
| NE | 0 | | 0 | | 0 | |
| ORR, n (%) | 22 | (51.2) | 12 | (57.1) | 34 | (53.1) |
| 80% CI [1] | 40.4, 61.9 | | 41.0, 72.2 | | 44.4, 61.7 | |
| 90% CI [1] | 37.7, 64.5 | | 37.2, 75.5 | | 42.1, 63.9 | |
| CBR, n (%) | 23 | (53.5) | 13 | (61.9) | 36 | (56.3) |

| | | | |
|---|---|---|---|
| 80% CI [1] | 42.6, 64.1 | 45.6, 76.4 | 47.5, 64.7 |

[1] Confidence interval is based on Clopper-Pearson confidence limits.

Results in each Cohort were also determined based on the subtype of B-cell non-Hodgkin lymphoma (B-NHL). Results for all fixed and single priming dose Cohorts by B-NHL subtype are summarized in Table 25. Abbreviations used in the below tables are as follows: DLBCL/HGBL=diffuse large B-cell lymphoma/high-grade B-cell lymphoma; FL=follicular lymphoma; MZL=marginal zone lymphoma; MCL=mantle cell lymphoma.

TABLE 25
All Fixed and Single Priming Cohorts by B-NHL Subtype

| | | | | | All |
|---|---|---|---|---|---|
| DLBCL/HGBL | FL | MZL | MCL | Other | Subtypes |
| (n = 16) | (n = 18) | (n = 3) | (n = 5) | (n = 1) | (n = 43) |

Best Overall Response, n (%)

| CR | 2 | (12.5) | 13 | (72.2) | 3 (100.0) | 1 (20.0) | 0 | 19 | (44.2) |
|---|---|---|---|---|---|---|---|---|---|
| PR | 1 | (6.3) | 1 | (5.6) | 0 | 1 (20.0) | 0 | 3 | (7.0) |
| MR | 0 | | 1 | (5.6) | 0 | 0 | 0 | 1 | (2.3) |
| SD | 0 | | 1 | (5.6) | 0 | 1 (20.0) | 0 | 2 | (4.7) |
| PD | 9 | (56.3) | 2 | (11.1) | 0 | 1 (20.0) | 1 (100.0) | 13 | (30.2) |
| NE | 0 | | 0 | | 0 | 0 | 0 | 0 | |
| ORR, n | 3 | (18.8) | 14 | (77.8) | 3 (100.0) | 2 (40.0) | 0 | 22 | (51.2) |
| (%) | | | | | | | | | |
| CBR, n | 3 | (18.8) | 15 | (83.3) | 3 (100.0) | 2 (40.0) | 0 | 23 | (53.5) |
| (%) | | | | | | | | | |

Results by subtype for the double step-up priming dose Cohorts 5d and 6d are shown in Tables 26 and 27.

TABLE 26
Cohort 5d (2400 ug dose) Results by B-NHL Subtype

| | | | | | All |
|---|---|---|---|---|---|
| DLBCL/HGBL | FL | MZL | MCL | Other | Subtypes |
| (n = 3) | (n = 5) | (n = 0) | (n = 0) | (n = 0) | (n = 8) |

Best Overall Response, n (%)

| CR | 1 | (33.3) | 4 | (80.0) | 0 | 0 | 0 | 5 | (62.5) |
|---|---|---|---|---|---|---|---|---|---|
| PR | 1 | (33.3) | 1 | (20.0) | 0 | 0 | 0 | 2 | (25.0) |
| MR | 1 | (33.3) | 0 | | 0 | 0 | 0 | 1 | (12.5) |
| SD | 0 | | 0 | | 0 | 0 | 0 | 0 | |
| PD | 0 | | 0 | | 0 | 0 | 0 | 0 | |
| NE | 0 | | 0 | | 0 | 0 | 0 | 0 | |
| ORR, n | 2 | (66.7) | 5 | (100.0) | 0 | 0 | 0 | 7 | (87.5) |
| (%) | | | | | | | | | |
| CBR, n | 3 | (100.0) | 5 | (100.0) | 0 | 0 | 0 | 8 | (100.0) |
| (%) | | | | | | | | | |

TABLE 27
Cohort 6d (7200 ug dose) Results by B-NHL Subtype

| | | | | | All |
|---|---|---|---|---|---|
| DLBCL/HGBL | FL | MZL | MCL | Other | Subtypes |
| (n = 10) | (n = 3) | (n = 0) | (n = 0) | (n = 0) | (n = 13) |

Best Overall Response, n (%)

| CR | 2 (20.0) | 2 | (66.7) | 0 | 0 | 0 | 4 | (30.8) |
|---|---|---|---|---|---|---|---|---|
| PR | 0 | 1 | (33.3) | 0 | 0 | 0 | 1 | (7.7) |
| MR | 0 | 0 | | 0 | 0 | 0 | 0 | |
| SD | 2 (20.0) | 0 | | 0 | 0 | 0 | 2 | (15.4) |
| PD | 5 (50.0) | 0 | | 0 | 0 | 0 | 5 | (38.5) |
| NE | 0 | 0 | | 0 | 0 | 0 | 0 | |
| ORR, n | 2 (20.0) | 3 | (100.0) | 0 | 0 | 0 | 5 | (38.5) |
| (%) | | | | | | | | |
| CBR, n | 2 (20.0) | 3 | (100.0) | 0 | 0 | 0 | 5 | (38.5) |
| (%) | | | | | | | | |

A target dose of 2400 μg or greater was found to have a significant ORR and CBR. A summary of results by B-NHL subtype for all doses 2400 μg or greater is provided in Table 28.

TABLE 28
Results for Doses 2400 ug or Greater by B-NHL Subtype

| | | | | | All |
|---|---|---|---|---|---|
| DLBCL/HGBL | FL | MZL | MCL | Other | Subtypes |
| (n = 27) | (n = 19) | (n = 2) | (n = 4) | (n = 1) | (n = 53) |

Best Overall Response, n (%)

| CR | 5 | (18.5) | 16 | (84.2) | 2 (100.0) | 1 (25.0) | 0 | 24 | (45.3) |
|---|---|---|---|---|---|---|---|---|---|
| PR | 2 | (7.4) | 2 | (10.5) | 0 | 1 (25.0) | 0 | 5 | (9.4) |
| MR | 1 | (3.7) | 0 | | 0 | 0 | 0 | 1 | (1.9) |
| SD | 2 | (7.4) | 0 | | 0 | 0 | 0 | 2 | (3.8) |
| PD | 12 | (44.4) | 1 | (5.3) | 0 | 1 (25.0) | 1 (100.0) | 15 | (28.3) |
| NE | 0 | | 0 | | 0 | 0 | 0 | 0 | |
| ORR, n | 7 | (25.9) | 18 | (94.7) | 2 (100.0) | 2 (50.0) | 0 | 29 | (54.7) |
| (%) | | | | | | | | | |
| CBR, n | 8 | (29.6) | 18 | (94.7) | 2 (100.0) | 2 (50.0) | 0 | 30 | (56.6) |
| (%) | | | | | | | | | |

Cytokine release after dosage administration was measured for the following cytokines Interleukins—IL-2, IL-6, IL-8 and IL-10; interferon gamma (INFγ); monocyte chemoattractant protein-1 (MCP-1); macrophage inflammatory protein-1 alpha (MIP-1α); tumor necrosis factor alpha (TNFα). Cytokine release was not observed for IL-2, IL-6, MIP-1α, INFγ and TNFα among any of the Cohorts. Some release of IL-8 and IL-10 was observed that generally resolved after 9-24 hours. Release of MCP-1 was seen in all Cohorts but, for most dosages, also resolved after 9-24 hours.

1. A method for treating B-cell non-Hodgkin lymphoma (B-NHL) in a patient in need, the method comprising administering to the patient a therapeutically effective amount of TNB-486 according to a 28-day treatment cycle, wherein the therapeutically effective amount of TNB-486 is from about 30 μg to about 30000 μg, optionally wherein the patient receives six treatment cycles.

2. The method according to claim 1, wherein the treatment cycle is repeated two or more times.

3. The method according to any one of the previous claims, wherein TNB-486 is administered to the patient as a monotherapy.

4. The method according to any one of the previous claims, wherein TNB-486 is administered by an intravenous infusion (IV).

5. The method according to any one of the previous claims, wherein the patient has received at least two prior lines of systemic therapy.

6. The method according to any one of the previous claims, wherein the patient's B-NHL disease is CD19-positive.

7. The method according to any one of the previous claims, wherein the patient has an Eastern Cooperative Oncology Group (ECOG) performance status of less than or equal to 2.

8. The method according to any one of the previous claims, wherein the patient has adequate bone marrow function.

9. The method according to any one of the previous claims, wherein the patient has an estimated glomerular filtration rate (eGFR) greater than or equal to 50 mL/min.

10. The method according to any one of the previous claims, wherein the patient has:
a total bilirubin of less than or equal to 1.5 times an upper limit of normal;
an aspartate aminotransferase (AST) of less than or equal to 3 times an upper limit of normal; and
an alanine amino transferase (ALT) of less than or equal to 3 times an upper limit of normal.

11. A method of improving an objective response rate (ORR) in a patient diagnosed with B-cell non-Hodgkin lymphoma, the method comprising administering to the patient a therapeutically effective amount of TNB-486 according to a 28-day treatment cycle, wherein the therapeutically effective amount of TNB-486 is from about 30 μg to about 30000 μg.

12. A method for improving an overall survival (OS) rate in a patient diagnosed with B-cell non-Hodgkin lymphoma, the method comprising administering to the patient a therapeutically effective amount of TNB-486 according to a 28-day treatment cycle, wherein the therapeutically effective amount of TNB-486 is from about 30 μg to about 30000 μg.

13. A method for improving a progression free survival (PFS) rate in a patient diagnosed with B-cell non-Hodgkin lymphoma, the method comprising administering to the patient a therapeutically effective amount of TNB-486 according to a 28-day treatment cycle, wherein the therapeutically effective amount of TNB-486 is from about 30 μg to about 30000 μg.

14. A method for improving a time to progression (TTP) in a patient diagnosed with B-cell non-Hodgkin lymphoma, the method comprising administering to the patient a therapeutically effective amount of TNB-486 according to a 28-day treatment cycle, wherein the therapeutically effective amount of TNB-486 is from about 30 μg to about 30000 μg.

15. A method for improving a time to response (TTR) in a patient diagnosed with B-cell non-Hodgkin lymphoma, the method comprising administering to the patient a therapeutically effective amount of TNB-486 according to a 28-day treatment cycle, wherein the therapeutically effective amount of TNB-486 is from about 30 μg to about 30000 μg.

16. A method for improving a duration of objective response (DOR) in a patient diagnosed with B-cell non-Hodgkin lymphoma, the method comprising administering to the patient a therapeutically effective amount of TNB-486 according to a 28-day cycle, wherein the therapeutically effective amount of TNB-486 is from about 30 μg to about 30000 μg.

17. A method for improving a clinical benefit rate (CBR) in a patient diagnosed with B-cell non-Hodgkin lymphoma, the method comprising administering to the patient a therapeutically effective amount of TNB-486 according to a 28-day cycle, wherein the therapeutically effective amount of TNB-486 is from about 30 μg to about 30000 μg.

18. The method according to any one of claims 11-17, wherein the improvement is at least about 5%, at least about 10%, at least about 15%, at least about 20%, at least about 25%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or at least about 100%.

19. The method according to any one of the previous claims, wherein the treatment cycle is modified to add more time between doses.

20. The method according to any one of claims 1-18, wherein the treatment cycle is modified by consistently eliminating one or more treatment cycles from a dosing regimen.

21. The method according to any one of claims 1-18, wherein the treatment cycle is modified to include a priming dose.

22. The method according to claim 21, wherein the priming dose is from about 150 μg to about 1500 μg.

23. The method according to claim 21, wherein the priming dose is from about 270 μg to about 1000 μg.

24. The method according to any one of claims 21-22, wherein the priming dose is administered at a first timepoint in the first treatment cycle, and a full dose is administered at all subsequent timepoints.

25. The method according to claim 23, wherein the priming dose is administered on day 1 of the first treatment cycle, a full dose is administered on day 15 of the first treatment cycle, and the full dose is administered on days 1 and 15 of all subsequent treatment cycles.

26. The method according to claim 21, wherein the treatment cycle is modified to include at least two priming doses.

27. The method according to claim 26, wherein the first priming dose is from about 150 μg to about 540 μg.

28. The method according to claim 26 or claim 27, wherein the second priming dose is from about 800 μg to about 1200 μg.

29. The method according to any one of claims 26-28, wherein the first priming dose is about 270 μg and the second priming dose is about 1000 μg.

30. The method according to any one of claims 26-29, wherein the first priming dose is administered on day 1 of the first treatment cycle, the second priming dose is administered on day 8 of the first treatment cycle, a full dose is administered on day 15 of the first treatment cycle, and the full dose is administered on days 1 and 15 of all subsequent treatment cycles.

31. The method according to claim 24, 25 or 30, wherein the full dose is equal to or less than 100% more than a full dose corresponding to a next lowest dose cohort.

32. The method according to claim 24, 25 or 30, wherein the full dose is equal to or less than 50% more than a full dose corresponding to a next lowest dose cohort.

33. The method according to claim 24, 25 or 30, wherein the full dose is equal to or less than 33% more than a full dose corresponding to a next lowest dose cohort.

34. The method according to any one of the previous claims, further comprising premedicating the patient prior to administration of TNB-486 with an agent that reduces a risk or severity of a hypersensitivity reaction.

35. The method according to claim 34, wherein the agent that reduces the risk or severity of a hypersensitivity reaction is selected from the group consisting of: dexamethasone, diphenhydramine, acetaminophen, ranitidine, tocilizumab, any equivalents thereof, or any combination thereof.

36. The method according to claim 34 or 45, wherein the agent that reduces the risk or severity of a hypersensitivity reaction is administered 15-60 minutes prior to administration of TNB-486.

37. The method according to any one of the preceding claims, wherein the therapeutically effective amount of TNB-486 is about 30 μg, 90 μg, 270 μg, 800 μg, 2400 μg, 7200 μg, 15000 μg, or 30000 μg.

38. The method according to any one of the preceding claims, wherein the TNB-486 is administered in combination with another chemotherapy.

39. The method according to claim 38, wherein the other chemotherapy is a combination of rituximab, cyclophosphamide, doxorubicin hydrochloride (Hydroxydaunomycin), vincristine sulfate (Oncovin), and prednisone (R-CHOP).

40. The method according to claim 39, wherein the R-CHOP is administered on day 1 of the first treatment cycle and then on day 1 of each subsequent treatment cycle.

41. The method according to claim 39, wherein each treatment cycle is 21 days long, wherein R-CHOP is administered on day 1 of a first treatment cycle, a first priming dose is administered on day 8 of the first treatment cycle, a second priming dose is administered on day 15 of the first treatment cycle, and wherein R-CHOP and a therapeutically effective amount of TNB-486 are administered on day 1 of a second treatment cycle.

42. The method according to claim 41, wherein R-CHOP and a therapeutically effective amount of TNB-486 are administered on day 1 of each subsequent treatment cycle.

43. The method according to any one of claims 21-42, wherein the patient has reduced cytokine release compared to a treatment cycle without a priming dose.

44. The method according to any one of claims 21-43, wherein the patient does not experience cytokine release syndrome or only experiences Grade 1 cytokine release syndrome.

45. The method according to claim 43 or claim 44, wherein the cytokine release syndrome comprises the release of IL-6 and/or TNF-α.

46. The method according to any one of the preceding claims, wherein the objective response rate (ORR) is 80% or above, the 6-month PFS rate is 90% or above, or the complete response (CR) rate is 90% or above.

47. The method of any previous claim, wherein TNB-486 is a bispecific molecule that binds to CD3 and human CD19, comprising: (i) a first polypeptide subunit comprising the amino acid sequence of SEQ ID NO: 18; (ii) a second polypeptide subunit comprising the amino acid sequence of SEQ ID NO: 11, wherein the first and second polypeptide subunits together form a first binding moiety that binds to human CD3; and (iii) a third polypeptide subunit that binds to human CD19, comprising the amino acid sequence of SEQ ID NO: 20.
